(19) United States
(12) Patent Application Publication
(10) Pub. No.: US 2007/0254859 A1
(43) Pub. Date: Nov. 1, 2007

(54) Compounds exhibiting efflux inhibitor activity and composition and uses thereof

(76) Inventors: Michael Fitzpatrick Wempe, Kingsport, TN (US); Kevin Joseph Edgar, Kingsport, TN (US); John Anthony Hyatt, Kingsport, TN (US); George Chester Zima, Kingsport, TN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/730,362

(22) Filed: Mar. 30, 2007

Related U.S. Application Data
(60) Provisional application No. 60/788,053, filed on Apr. 3, 2006.

(51) Int. Cl. A61K 31/58; C07J 17/00

(52) U.S. Cl. 514/172; 540/114

(57) ABSTRACT

At least one compound chosen from compounds of Formula I:

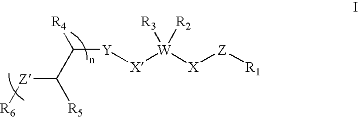

a pharmaceutically acceptable salt or ester thereof, a solvate thereof, a chelate thereof, a non-covalent complex thereof, a prodrug thereof, and mixtures of any of the foregoing, wherein: n is a number from 1 to 900, wherein the individual units may be the same or different; W is chosen from alkyl, substituted alkyl, cycloalkyl, substituted cycloalkyl, aryl, substituted aryl, aralkyl and substituted aralkyl; each of R₂, R₃, R₄ and R₅ is independently chosen from —H, alkyl, substituted alkyl, cycloalkyl, substituted cycloalkyl, aryl, substituted aryl, aralkyl and substituted aralkyl; Z′ is chosen from —O—, —N—, —NO—, —NR₄—, —S—, —SO— and —SO₂—, wherein R₄ is defined as above; each of X, X′, Y and Z is independently chosen from —CR₄R₅—, —NH—, —NR₄—, —NO—, —O—, —NOR₄—, —S—, —SO—, —SO₂—, wherein R₄ and R₅ are defined as above; R₁ is chosen from a tocopherol, a steroid and a flavonoid; and R₆ is chosen from any R₁, alkyl, substituted alkyl, cycloalkyl, substituted cycloalkyl, aryl, substituted aryl, aralkyl and substituted aralkyl.

This application claims the benefit of U.S. provisional application no.60/788,053, filed Apr. 3, 2006, which is incorporated herein by reference.

Water-soluble vitamin E-active polyethylene glycol esters of tocopheryl acid such as succinates were developed to provide water-soluble molecules having high vitamin E activity via either oral or parenteral administration. Examples include the polyethylene glycol acid succinate of α-tocopherol, known as d-α-tocopheryl polyethylene glycol succinate (TPGS). U.S. Pat. No. 2,680,749 discloses TPGS molecules in which the polyethylene glycols have average molecular weights of 400, 1000, and those varying between 600 and 6000.

TPGS molecules where the polyethylene glycol has an average molecular weight. (MW) of about 1000 (TPGS 1000; available from Eastman Chemical Company, Kingsport, Tenn.) are currently used in oral pharmaceutical applications to enhance the bioavailability of various drugs. Due to the amphiphilic nature of TPGS 1000, incorporating TPGS 1000 into pharmaceutical formulations enhances oral bioavailability by solubilizing some hydrophobic drugs. TPGS 1000 is also believed to influence one or more transporter proteins, one example of which is P-glycoprotein (P-gp), an enzyme that acts as a cellular efflux pump. Therefore, TPGS 1000 may contribute to oral bioavailability enhancement by influencing efflux of some drugs.

TPGS 1000 possesses mild to moderate efflux inhibition properties. Additional compounds, such as cyclosporine (CSA), possess greater efflux inhibition properties but also have serious toxic properties and exhibit clinical side effects. From a clinical perspective, it would be a technological advancement to have efflux inhibitors that are non-toxic like TPGS 1000, but that possess greater efflux inhibition properties.

Provided herein is at least one compound chosen from compounds of Formula I: 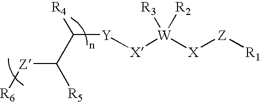
a pharmaceutically acceptable salt or ester thereof, a solvate thereof, a chelate thereof, a non-covalent complex thereof, a prodrug thereof, and mixtures of any of the foregoing, wherein:

n is a number from 1 to 900, wherein the individual units may be the same or different;

W is chosen from alkyl, substituted alkyl, cycloalkyl, substituted cycloalkyl, aryl, substituted aryl, aralkyl and substituted aralkyl;

each of R₂, R₃, R₄ and R₅ is independently chosen from —H, alkyl, substituted alkyl, cycloalkyl, substituted cycloalkyl, aryl, substituted aryl, aralkyl and substituted aralkyl;

Z′ is chosen from —O—, —N—, —NO—, —NR₄—, —S—, —SO— and —SO₂—, wherein R₄ is defined as above;

each of X, X′, Y and Z is independently chosen from —CR₄R₅—, —NH—, —NR₄—, —NO—, —O—, —NOR₄—, —S—, —SO—, —SO₂—, wherein R₄ and R₅ are defined as above;

R₁ is chosen from a tocopherol, a steroid and a flavonoid; and

R₆ is chosen from any R₁, alkyl, substituted alkyl, cycloalkyl, substituted cycloalkyl, aryl, substituted aryl, aralkyl and substituted aralkyl.

Also provided herein is at least one compound chosen from compounds of Formula II: 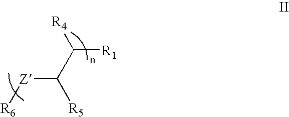
a pharmaceutically acceptable salt or ester thereof, a solvate thereof, a chelate thereof, a non-covalent complex thereof, a prodrug thereof, and mixtures of any of the foregoing, wherein:

n is a number from 1 to 900, wherein the individual units may be the same or different;

each of R₄ and R₅ is independently chosen from —H, alkyl, substituted alkyl, cycloalkyl, substituted cycloalkyl, aryl, substituted aryl, aralkyl and substituted aralkyl;

Z′ is chosen from —O—, —N—, —NO—, —NR₄—, —S—, —SO— and —SO₂—, wherein R₄ is defined as above;

R₁ is chosen from a tocopherol, a steroid and a flavonoid; and

R₆ is chosen from any R₁, alkyl, substituted alkyl, cycloalkyl, substituted cycloalkyl, aryl, substituted aryl, aralkyl and substituted aralkyl.

As used herein, the term “tocopherol” refers to a family of natural or synthetic compounds known by generic names such as vitamin E. Tocol and tocotrienol compounds are included in this family. Tocotrienols are similar in structure to the tocopherols with the exception of three double bonds in the phytyl side chain. Formula III illustrates a compound of the present invention wherein R₁ is the general structure of a tocopherol. Formula IV illustrates the general formula of the tocopherol in isolation from the rest of the compound. R₁

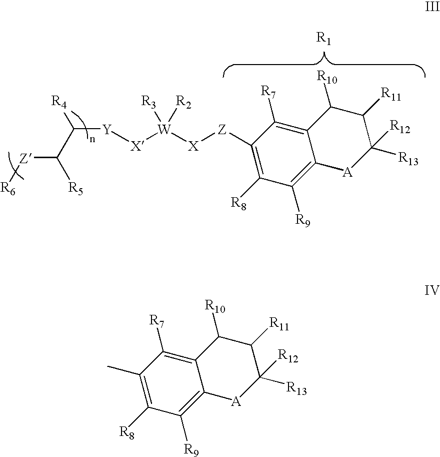

Tocopherols suitable for use in the present invention include compounds of Formula IV wherein each of R₇, R₈, R₉, R₁₀, R₁₁, R₁₂ and R₁₃ is independently chosen from —H, alkyl, substituted alkyl, cycloalkyl, substituted cycloalkyl, aryl, substituted aryl, aralkyl and substituted aralkyl; and A is chosen from —CR₄R₅—, —O—, —NH—, —NR₄—, —NO—, —NOR₄—, —S—, —SO— and —SO₂—, wherein each of R₄ and R₅ is independently chosen from —H, alkyl, substituted alkyl, cycloalkyl, substituted cycloalkyl, aryl, substituted aryl, aralkyl and substituted aralkyl.

In certain embodiments, the tocopherol is a compound of Formula IV wherein each of R₇, R₈ and R₉ is independently chosen from —H, C₁₋₁₂ n-alkyl, substituted C₁₋₁₂ n-alkyl, C₃₋₁₂ branched alkyl, substituted C₃₋₁₂ branched alkyl, C₃₋₈ cycloalkyl, substituted C₃₋₈ cycloalkyl, aryl, substituted aryl, aralkyl and substituted aralkyl; each of R₁₀, R₁₁, R₁₂ and R₁₃ is independently chosen from —H, C₁₋₃₀ n-alkyl, substituted C₁₋₃₀ n-alkyl, C₃₋₄₈ branched alkyl, substituted C₃₋₄₈ branched alkyl, C₃₋₈ cycloalkyl, substituted C₃₋₈ cycloalkyl, aryl, substituted aryl, aralkyl and substituted aralkyl; and A is chosen from —CR₄R₅—, —NH—, —NR₄—, —NO—, —NOR₄—, —S—, —SO— and —SO₂—, wherein each of R₄ and R₅ is independently chosen from —H, C₁₋₁₂ n-alkyl, substituted C₁₋₁₂ n-alkyl, C₃₋₁₂ branched alkyl, substituted C₃₋₁₂ branched alkyl, C₃₋₈ cycloalkyl, substituted C₃₋₈ cycloalkyl, aryl, substituted aryl, aralkyl and substituted aralkyl.

As used herein, the term “steroid” refers to a broad family of natural or synthetic compounds known by generic names as steroids where three cyclohexane rings are fused to each other and usually, but not necessarily, by trans-ring junctures. The fourth ring is a cyclopentane to afford a tetracyclic structure generically labeled as A, B, C, D; however, these ring systems may vary. Many steroids have methyl groups attached at C-10 and C-13 and oxygen at C-3 and C17. In addition, longer side chains may be found at C-17; K. Peter C. Vollhardt, Organic Chemistry (1987), W. H. Freeman and Company, New York. Formula V illustrates a compound of the present invention wherein R₁ is the general structure of a steroid.

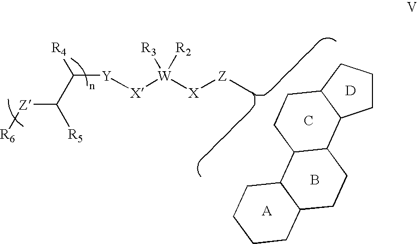

Examples of steroids include, but are not limited to: anabolic steroids (androisozazole, androstenediol, bolasterone, bolandiol, clostebol, ethylestrenol, formyldienolone, 4-hydroxy-19-nortestosterone, methandriol, methyltrienolone, methenolone, methyltrienolone, nandrolone, norbolethone, oxymesterone, stanozolol, stenbolone, trenbolone, and the like), androgens (boldine, boldenone, fluoxymesterone, mestanolone, mesterolone, methandrostenolone, 17-methyltestosterone, 17α-methyltestosterone 3-cyclopentyl enol ether, norethandrolone, normethandrone, oxandrolone, oxymesterone, oxymetholone, prasterone, stanolone, testosterone, tibolone, tiomesterone, mibolerone, and the like), estrogens (equilenin, equilin, estradiol, estradiol 17β-cypionate, estriol, estrone, ethinyl estradiol, mestranol, moxestrol, mytatrienediol, quinestradiol, quinestrol, calusterone, and the like), anti-allergic and anti-inflammatory (beclomethasone, budesonide, dexamethasone, flunisolide, triamcinolone acetonide, meprednisone, 21-acetoxypregnenolone, alclometasone, algestone, amcinonide, betamethasone, chloroprednisone, clobetasol, clobetasone, clocortolone, cloprednol, corticosterone, cortisone, cortisone 21 β-cyclopentaneproprionate, cortisone phosphate, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocotolone, fluorometholone, fluperolone acetate, fluprednidene acetate, fluprednisolone, flurandrenolide, formocortal, halcinonide, halometasone, halopredone acetate, hydrocortamate, hydrocortisone, isoflupredone, mazipredone, medrysone, meprednisone, methylprednisolone, mometasone furoate, prednicarbate, prednisolone, prednisone, prednival, prednylidene, tixocortol, tomatidine, triamcinolone, triamcinolone acetonide, and the like), anti-neoplastic (epitiostanol, calusterone, estramustine, melengestrol, prednimustine, and the like), anesthetics (alfadolone acetate, alfaxalone, and the like), bile acids (cholic acid, deoxy-cholic acid, litho-cholic acid, ursodeoxy-cholic acid, dehydro-cholic acid, cheno-deoxy-cholic acid, glycocholic acid, taurocholic acid, and the like), mineralcorticoid (aldosterone, deoxycorticosterone, fludrocortisone, and the like), cholesterol (allocholesterol, azacosterol, campesterol, cephalosporin P1, CHAPS, chenodiol, cholanic acid, cholestanol, chondrillasterol, chonemorphine, coprosterol, 7-dehydrocholesterol, dehydroergosterol, 7-dehydrositosterol, desmosterol, dihydrotachysterol, dinosterol, ecdysones, epiandrosterone, epicholesterol, epicholestanol, ergostanol, α-ergostenol, β-ergostenol, γ-ergostenol, etiocholanic acid, fucosterol, 24-hydroxycholesterol, 25-hydroxycholesterol, hydroxydione, hyodeoxycholic acid, lanosterol, lumisterol, neoergosterol, norcholanic acid, α₁-sitosterol, β-sitosterol, γ-sitosterol, α-spinasterol, stigmastanol, stigmasterol, and the like), progestins (desogestrel, dimethisterone, norgestrel, progesterone, norgesterone, norgestimate, medroxyprogesterone, melengestrol, allylesternol, altrenogest, anagestone, ethisterone, flurogestone acetate, gestodene, gestonorone caproate, haloprogesterone, 17-hydroxy-16-methylene-Δ⁶-progesterone, 17α-hydroxyprogesterone, norethynodrel, normethadrone, norvinisterone, pentagestrone, periplgenin, peruvoside, and the like), anti-ulcerative (acetoxime, acetoxolone, carbenoxolone, and the like), others (acacic acid, β-boswellic acid, brassinolide, cafestol, calcitriol, calcifediol, calotropin, cascarillin, cassaidine, cassaine, cassamine, caenothic acid, cevadine, cevine, chlorogenin, cimigenol, cinobufotalin, colforsin, columbin, conessine, convallamarogenin, coumingine, cucurbitacins, cyclobuxine, cyclopregnol, cynanchogenin, danazol, 11-dehydrocorticosterone, desatrine, deslanoside, 11-desoxy-17-hydroxycorticosterone, dichlorisbne, doisynoestrol, doisynolic acid, 16-epiestriol, epimetrol, erythrophlamine, erythrophleine, escigenin, escin, α-estradiol, ethynodiol, fungisterol, funtumine, furazabol, fusidic acid, gamabufotalin, gentrogenin, germine, gratiogenin, grindelic acid, gypsogenin, hecogenin, hederagenin, α-hederin, hellebrin, helvoic acid, hexahydroequilenin, -holarrhenine, hydrallostane, 4-hydroxy-19-nortestosterone, imperialine, isoestradiol, 8-isoestrone, isopimaric acid, isopyrocalciferol, isorubijervine, jervine, kurchessine, kurcholessine, levopimaric acid, lupeol, lynesternol, mifepristone, neriifolin, norethindrone, oleandrin, oleanolic acid, ouabagenin, ouabain, oxendolone, palustric acid, pavoninin-5, pimaric acid, ponasterone A, pregnanediol, pregnan-3α-ol-20-one, 4-pregnene-20,21-diol-3,11-dione, 4-pregnene-11β,17α,20β,21-tetrol-3-one, pregnenolone, proscillaridin, protoverine, pseudohecogenin, pyrocalciferol, quillaic acid, quinovic acid, resibufogenin, rubijervine, ruscogenin, sabadine, samandarine, sarmentogenin, sarsasapogenin, sarverogenin, scillaren, scillarenin, scilliroside, senegenin, solanidine, solanocapsine, solasodine, steviol, stevioside, strophanthidin, sulphurenic acid, suprasterol II, tachysterol, tanghinigenin, taraxasterol, taraxerol, taxodione, tibolone, tigogenin, trilostane, uranediol, ursodiol, uzarin, veralkamine, veratramine, vernadigin, verticine, vitamin D₂, vitamin D₃, Vitamin D₄, withaferin A, zygadenine, norethindrone, norethynodrel, oxendolone, androsterone, androst-16-en-3-ol, cyproterone, allotetrahydrocortisone, androstane-3β,11β-diol-17-one, androst-16-en-3-ol, androsterone, antheridiol, α-antiarin, aphidicolin, batrachotoxin, batrachotoxinin A, betulin, bufalin, bufogenin B, bufotalin, bufotoxin, cerberoside, cortol, cortolone, corybulbine, diginatigenin, digitalin, digitogenin, digitoxigenin, digoxigenin, dihydroequilin, diosgenin, ergosterol, gestrinone, gitogenin, gitoxigenin, gitoxin, and the like)

In some embodiments, R₁ in the at least one compound of Formula I may be a steroid. In other embodiments, the steroid may be a bile salt. In yet other embodiments, the steroid may be cholesterol or a derivative thereof. In some embodiments the steroid may be chosen from cholic acid, deoxy-cholic acid, litho-cholic acid, ursodeoxy-cholic acid, dehydro-cholic acid, cheno-deoxy-cholic acid, glycocholic acid, taurocholic acid, cholesterol, allocholesterol, campesterol, cholanic acid, cholestanol, coprosterol, 7-dehydrocholesterol, dehydroergosterol, 7-dehydrositosterol, desmosterol, lanosterol, α₁-sitosterol, β-sitosterol, γ-sitosterol, stigmastanol, and stigmasterol.

As used herein, the term “flavonoid” refers to a family of natural or synthetic compounds known by generic names as flavanones, isoflavones, flavones, chromones, coumarins, chalcones and the like. Formula VI illustrates a compound of the present invention wherein R₁ is the general structure of a flavonoid.

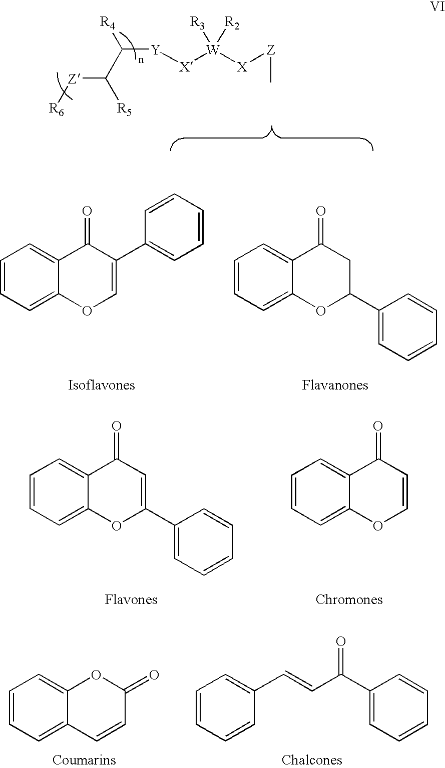

Examples of flavonoids include, but are not limited to: flavanones: (ampelopsin, eriodictyol, fustin, hesperetin, homoeriodictyol, sakuranetin, bavachinin A, didymin, dihydrorobinetin, 5,7-dihydroxyflavan one, 6,2′-dihydroxyflavanone, 5,4′-dihydroxy-7-O-methoxyflavanone, 5,7-dihydroxy-3′,4′,5′-trimethoxyflavanone, 5,7-dimethoxy-4′-hydroxyflavanone, eriocitrin, eriodictyol-7-O-glucoside, flavanomarein, hesperidiri, homoeriodictyol, 2′-hydroxyflavanone, 3′-hydroxyflavanone, 4′-hydroxyflavanone, 6-hydroxyflavanone, 7-hydroxyflavanone, 4′-hydroxy-3′-methoxyflavanone, 5-hydroxy-7-methoxyflavanone, (S)-7-hydroxy-5-methoxyflavanone, isosakuranetin, liquiritigenin, naringenin, naringenin-7-O-glucoside, naringin, narirutin, neoeriocitrin, neohesperidin, 3,5,7,3′,4′-pentahydroxyflavanone, poncirin, silybin, 3,7,3′,4′-tetrahydroxyflavanone, and the like), flavones (acacetin, amentoflavone, apigetrin, apigenin, apigenin-7-O-glucoside, apiin, baicalein, baicalin, 3′-benzyloxy-5,7-dihydroxy-3,4′-dimethoxyflavone, 8-carboxy-3-methylflavone, chrysoeriol, cupressuflavone, centaurein, chrysergonic acid, datiscetin, datiscoside, dimefline, diosmetin, diosmin, 3,7-dihydroxy-3′,4′-dimethoxyflavone, 2′,3′-dihydroxyflavone, 2′,4′-dihydroxyflavone, 3,2′-dihydroxyflavone, 3,3′-dihydroxyflavone, 3,4′-dihydroxyflavone, 3′,4′-dihydroxyflavone, 3,5-dihydroxyflavone, 3,6-dihydroxyflavone, 3,7-dihydroxyflavone, 5,2′-dihydroxyflavone, 5,3′-dihydroxyflavone, 5,4′-dihydroxyflavone, 5,6-dihydroxyflavone, 5,7-dihydroxyflavone, 6,2′-dihydroxyflavone, 6,3′-dihydroxyflavone, 6,4′-dihydroxyflavone, 6,7-dihydroxyflavone, 7,2′-dihydroxyflavone, 7,3′-dihydroxyflavone, 7,4′-dihydroxyflavone, 7,8-dihydroxyflavone, 5,4′-dihydroxy-7-methoxyflavone, 5,6-dihydroxy-7-methoxyflavone, 3′,4′-dihydroxy-α-naphthoflavone, 3′,4′-dihydroxy-β-naphthoflavone, 5,8-dihydroxy-3,7,3′,4′-tetramethoxyflavone, 3,7-dihydroxy-3′,4′,5′-trimethoxyflavone, 5,3′-dihydroxy-6,7,4′-trimethoxyflavone, 5,7-dihydroxy-3′,4′,5′-trimethoxyflavone, 2′,3′-dimethoxy-3-hydroxyflavone, 2′,4′-dimethoxy-3-hydroxyflavone, 6,2′-dimethoxy-3-hydroxyflavone, 6,3′-dimethoxy-3-hydroxyflavone, 6,4′-dimethoxy-3-hydroxyflavone, 7,2′-dimethoxy-3-hydroxyflavone, 7,3′-dimethoxy-3-hydroxyflavone, 7,4′-dimethoxy-3-hydroxyflavone, 2′,4′-dimethoxy-3-hydroxy-6-methylflavone, 3′,4′-dimethoxy-3-hydroxy-6-methylflavone, 3′,5′-dimethoxy-3,5,7,4′-tetrahydroxyflavone, 3,4′-dimethoxy-5,7,3′-trihydroxyflavone, 6,7-dimethoxy-5,3′,4′-trihyroxyflavone, eupatorin-5-methylether, fisetin, fortunellin, galangin, gardenin, geraldol, gossypetin, gossypin, hinokiflavone, homoorientin, 6-hydroxyapigenin, 2′-hydroxyflavone, 3-hydroxyflavone, 3′-hydroxyflavone, 4′-hydroxyflavone, 5-hydroxyflavone, 6-hydroxyflavone, 7-hydroxyflavone, 3-hydroxy-2′-methoxyflavone, 3-hydroxy-3′-methoxyflavone, 3-hydroxy-4′-methoxyflavone, 3-hydroxy-5-methoxyflavone, 3-hydroxy-6-methoxyflavone, 3-hydroxy-7-methoxyflavone, 4′-hydroxy-3′-methoxyflavone, 4′-hydroxy-5-methoxyflavone, 4′-hydroxy-6-methoxyflavone, 4′-hydroxy-7-methoxyflavone, 5-hydroxy-2′-methoxyflavone, 5-hydroxy-3′-methoxyflavone, 5-hydroxy-4′-methoxyflavone, 5-hydroxy-7-methoxyflavone, 6-hydroxy-2′-methoxyflavone, 6-hydroxy-3′-methoxyflavone, 6-hydroxy-4′-methoxyflavone, 6-hydroxy-7-methoxyflavone, 7-hydroxy-2′-methoxyflavone, 7-hydroxy-3′-methoxyflavone, 7-hydroxy-4′-methoxyflavone, 8-hydroxy-7-methoxyflavone, 3-hydroxy-4′-methoxy-6-methylflavone, 3-hydroxy-6-methylflavone, 3′-hydroxy-6-methylflavone, 4′-hydroxy-6-methylflavone, 7-hydroxy-3-methylflavone, 7-hydroxy-5-methylflavone, 2′-hydroxy-α-naphthoflavone, 2′-hydroxy-β-naphthoflavone, 3′-hydroxy-α-naphthoflavone, 3′-hydroxy-β-naphthoflavone, 4′-hydroxy-α-naphthoflavone, 4′-hydroxy-β-naphthoflavone, 3′-hydroxy-5,6,7,4′-tetramethoxyflavone, 3-hydroxy-7,3′,4′,5′-tetramethoxyflavone, 3-hydroxy-3′,4′,5′-tetramethoxyflavone, 3-hydroxy-6,2′,3′-trimethoxyflavone, 3-hydroxy-6,2′,4′-trimethoxyflavone, 3-hydroxy-6,3′,4′-trimethoxyflavone, 3-hydroxy-7,2′,3′-trimethoxyflavone, 3-hydroxy-7,2′,4-trimethoxyflavone, hyperoside, isohamnetin, isohamnetin-3-O-glucoside, isohamnetin-3-O-rutinoside, isorhoifolin, isovitexin, kaempferide, kaempferol, kaempferol-3-O-glucoside, kaempferol-7-0-neohesperidoside, kaempferol-3-O-rutinoside, kaempferol-3,7,4′-trimethylether, linarin, luteolin, luteolin-7,3′-di-O-glucoside, luteolin-4′-O-glucoside, luteolin-7-O-glucosdie, maritimein, 4′-methoxyflavonol, 6-methoxyluteolin, neodiosmin, orientin, peltatoside, 3,7,3′,4′,5′-pentahydroxyflavone, 5,7,3′,4′,5′-pentahydroxyflavone, quercetin 3-O-glucopyranoside, quercetin-3-O-glucose-6″-acetate, quercetin-3,7,3′,4′-tetramethyl ether, rhoifolin, spiraeoside, sulfuretin, syringetin-3-O-galactoside, syringetin-3-O-glucoside, tamarixetin, 3,6,2′,3′-tetrahydroxyflavone, 3,6,2′,4′-tetrahydroxyflavone, 3,6,3′,4′-tetrahydroxyflavone, 7,3′,4′,5′-tetrahydroxyflavone, 7,8,3′,4′-tetrahydroxyflavone, 3,5,3′,4′-tetrahydroxy-7-methoxyflavone, 3,3′,4′-trihydroxyflavone, 3,6,2′-trihydroxyflavone, 3,6,3′-trihydroxyflavone, 3,6,4′-trihydroxyflavone, 3,7,3′-trihydroxyflavone, 3,7,4′-trihydroxyflavone, 5,3′,4′-trihydroxyflavone, 5,7,2′-trihydroxyflavone, 5,7,8-trihydroxyflavone, 6,2′,3′-trihydroxyflavone, 6,3,4′-trihydroxyflavone, 6,7,3′-trihydroxyflavone, 7,3′,4′-trihydroxyflavone, 7,8,2′-trihydroxyflavone, 7,8,3′-trihydroxyflavone, 7,8,4′-trihydroxyflavone, 3,5,7-trihydroxy-3′,4′,5′-trimethoxyflavone, vitexin, efloxate, eupatorin, fisetin, flavoxate, galangin, gardenins, isoquercitrin, morin, morindin, myricetin, oroxylin A, pectolinarigenin, quercetagetin, quercetin, rhamnetin, robinin, rutin, saponarin, scoparin, scutellarein, and the like), isoflavones: (baptigenin, daidzein, formononetin, genistein, ipriflavone, irigenin, irisolone, isoflavone, naringenin, pratensein, pseudobaptigenin, prunetin, sophorabioside, sophoricoside, tectorigenin, tetrahydrocannabinols, tetrahydrocortisone, 4′-acetoxy-7-hydroxy-6-methoxyisoflavone, biochanin A, 4′-chloro-7-hydroxy-8-methylisoflavone, daidzin, 3′,4′-dimethoxy-7-hydroxyisoflavone, 6,4′-dimethoxy-7-hydroxyisoflavone, 7,4′-dimethoxy-5-hydroxyisoflavone, equol, genistin, glycitein, glycitein, glycitin, 7-hydroxyisoflavone, 7-hydroxy-6-methoxyisoflavone, 7-hydroxy-4′-methoxy-8-methylisoflavone, 7-hydroxy-8-methylisoflavone, puerarin, sissotrin, 6,7,4′-trihydroxyisoflavone, 7,3′,4′-trihydroxyisoflavone and the like), chromones: (bergapen(e), bucumolol, chromocarb, cromolyn, tricromyl, 6-bromochromone-2-carboxylic acid, 7-chlorochromone-2-sulfonyl-, 6-chloro-7-methylchromone-2-carboxylic acid, chromone-2-carboxylic acid, chromone-3-carboxylic acid, 6-methylchromone-2-carboxylic acid, and the like), coumarins: (acenocoumarol, brodifacoum, bromadiolone, cichoriin, coumachlor, coumafuryl, coumarin, coumarin-3-carboxylic acid, cyclocumarol, daphnetin, daphnin, dicumarol, esculetin, esculin, ethyl biscoumacetate, ethylidene dicoumarol, folescutol, fraxetin, fraxin, fraxidin, hymecromone, hymecromone O,O-diethyl phosphorothioate, limettin, mercumallylic acid, ostruthin, ostruthol, phenprocoumon, scopoletin, tioclomarol, warfarin, 8-acetyl-7-hydroxycoumarin, 8-acetyl-6-hydroxy-7-methoxycoumarin, 8-acetyl-7-hydroxy-4-methylcoumarin, 3-aminocoumarin, 4-amino-9-methoxy psoralen, 7-amino-4-methylcoumarin, 4-benzyl-7-hydroxy-3-phenylcoumarin, bergaptol, 6-bromo-8-chloro-4-hydroxycoumarin, 8-bromo-6-chloro-4-hydroxycoumarin, 6-bromo-8-chloro-3(4′-hydroxyphenyl)-4-methylcoumarin, 8-bromo-6-chloro-3(4′-hydroxyphenyl)-4-methylcoumarin, 6-bromo-4-hydroxycoumarin, 3(3′-bromophenyl)-7-hydroxycoumarin, 3(4′-bromophenyl)-7-hydroxycoumarin, 3(3′-bromophenyl)-7-hydroxy-4-methylcoumarin, 3(4′-bromophenyl)-6-hydroxy-4-methylcoumarin, 3(4′-bromophenyl)-7-hydroxy-4-phenylcoumarin, 6-chloro-3(3′,4′-dimethoxyphenyl)-7-hydroxy-4-methylcoumarin, 6-chloro-4-hydroxycoumarin, 6-chloro-7-hydroxycoumarin, 6-chloro-4-hydroxy-7-methoxycoumarin, 6-chloro-4-hydroxy-7-methoxy-3(4′-methoxyphenyl)coumarin, 3-chloro-7-hyroxy-4-methylcoumarin, 6-chloro-4-hyroxy-7-methylcoumarin, 6-chloro-7-hydroxy-4-methylcoumarin, 6-chloro-4-hydroxy-7-methyl-3-nitrocoumarin, 6-chloro-7-hydroxy-4-methyl-3-phenylcoumarin, 6-chloro-3(4′-hydroxyphenyl)-4-methylcoumarin, coumarin-6-sulfonyl-, 3-cyano-7-hydroxycoumarin, 3-cyano-7-hydroxy-4-methylcoumarin, dalbergin, 6,8-dichloro-4-hydroxycoumarin, 3(2′,4′-dichlorophenyl)-7-ethoxy-4-phenylcoumarin, 3(2′,4′-dichlorophenyl)-6-hydroxy-4-methylcoumarin, 3(2′,4′-dichlorophenyl)-7-hydroxy-4-methylcoumarin, 3(2′,4′-dichlorophenyl)-7-hydroxy-4-phenylcoumarin, 7,8-dihydroxycoumarin, 6,7-dihyroxycoumarin- 4-acetic acid, 5,7-dihydroxy-4-methylcoumarin, 6,7-dihydroxy-4-methylcoumarin, 7,8-dihydroxy-4-methylcoumarin, 5,7-dihydroxy-4-methylcoumarin-3-acetic acid, 7,8-dihydroxy-4-methylcoumarin-3-acetic acid, 6,7-dihydroxy-4-phenylcoumarin, 3(3′4′-dimethoxyphenyl)-7-hydroxycoumarin, 3(3′4′-dimethoxyphenyl)-6-hydroxy-4-methylcoumarin, 3(3′4′-dimethoxyphenyl)-7-hydroxy-4-methylcoumarin, 6,8-dimethyl-4-hydroxycoumarin, 6,7-dimethyl-4-hydroxycoumarin, 3,4-diphenyl-7-hydroxycoumarin, 6-ethoxy-3(4′-hydroxyphenyl)-4-methylcoumarin, 6-ethyl-4-hydroxycoumarin, ethyl-7-hydroxycoumarin-4-carboxylate, 6-fluoro-4-hydroxycoumarin, 3-hydroxycoumarin, 4-hydroxycoumarin, 7-hydroxycoumarin, 7-hydroxycoumarin-4-acetic acid, 7- hydroxycoumarin-3-carboxylic acid, 7-hydroxy-3(4′-hydroxyphenyl)-4-methylcoumarin, 4-hydroxy-6-methoxycoumarin, 4-hydroxy-7-methoxycoumarin, 6-hydroxy-7-methoxycoumarin, 7-hydroxy-6-methoxycoumarin, 8-hydroxy-7-methoxycoumarin, 4-hydroxy-7-methoxy-3(4′-methoxyphenyl)coumarin, 7-hydroxy-4-methoxymethylcoumarin, 4-hydroxy-7-methoxy-3-phenylcoumarin, 7-hydroxy-3(2′-methoxyphenyl)coumarin, 7-hydroxy-3(4′-methoxyphenyl)coumarin, 6-hydroxy-3(4′-methoxyphenyl)-4-methylcoumarin, 7-hydroxy-3(4′-methoxyphenyl)-4-methylcoumarin, 7-hydroxy-3(4′-methoxyphenyl)-4-phenylcoumarin, 4-hydroxy-6-methylcoumarin, 4-hydroxy-7-methylcoumarin, 6-hydroxy-4-methylcoumarin, 7-hydroxy-4-methylcoumarin, 7-hydroxy-4-methylcoumarin-3-acetic acid, 4-hydroxy-7-methyl-3-nitrocoumarin, 6-hydroxy-4-methyl-3-phenylcoumarin, 7-hydroxy-4-methyl-3-phenylcoumarin, 7-hydroxy-4-methyl-3(2-thiophenyl)coumarin, 4-hydroxy-3-nitrocoumarin, 7-hydroxy-3-phenylcoumarin, 7-hydroxy-4(2-pyridyl)coumarin, 7-hydroxy-4-(3-pyridyl)coumarin, 7-hydroxy-4-(4-pyridyl)coumarin, 7-hydroxy-4-trifluoromethylcoumarin, 7-hydroxy-4-(3-trifluoromethylphenyl)coumarin, (7-methoxycoumarin-4-yl)-acetic acid, methyl-6,7-dihydroxycoumarin-4-acetate, methyl-6,7-dimethoxycoumarin-4-acetate, methyl-7-hydroxycoumarin-4-acetate, N-succinimidyl-7-hydroxy-4-methyl-3-coumarinyl acetate, xanthotoxol, and the like), chalcones: (sofalcone, phloridzin, phloretin, 4-benzyloxy-2′-hydroxy-3,4′,6′-trimethoxychalcone, 5′-bromo-3′-chloro-2,5-dimethoxy-2′-hydroxychalcone, 3′-bromo-5′-chloro-2′-hydroxychalcone, 5′-bromo-3′-chloro-2′-hydroxychalcone, 5′-bromo-4-chloro-2′-hydroxychalcone, 3′-bromo-5′-chloro-2′-hydroxy-4-methoxychalcone, 5′-bromo-3′-chloro-2′-hydroxy-4-methoxychalcone, 3′-bromo-5′-chloro-2′-hydroxy-3,4,5-trimethoxychalcone, 5′-bromo-3′-chloro-2′-hydroxy-3,4,5-trimethoxychalcone, 5-bromo-2,2′-dihydroxy-4′,6′-dimethoxychalcone, 5′-bromo-4,2′-dihydroxy-3-methoxychalcone, 3-bromo-4′,6′-dimethoxy-2′-hydroxychalcone, 5′-bromo-2,5-dimethoxy-2′-hydroxychalcone, 5′-bromo-2′-hydroxy-4-methoxychalcone, 5-bromo-2-hydroxy-2′,4′,6′-trimethoxychalcone, 5′-bromo-2′-hydroxy-2′,4′,6′-trimethoxychalcone, butein, 5′-chloro-4,2′-dihydroxy-3-methoxychalcone, 5′-chloro-4,2′-dihydroxy-3-methoxy-4′-methylchalcone, 2-chloro-4′,6′-dimethoxy-2′-hydroxychalcone, 4-chloro-4′,6′-dimethoxy-2′-hydroxychalcone, 5′-chloro-2,5-dimethoxy-2′- hydroxychalcone, 5′-chloro-3,4-dimethoxy-2′-hydroxychalcone, 5′-chloro-2,5-dimethoxy-2′-hydroxy-4′-methylchalcone, 3′-chloro-4-fluoro-2′-hydroxychalcone,4-chloro-5′-fluoro-2′-hydroxychalcone, 3′-chloro-2′-hydroxychalcone, 4-chloro-2′-hydroxychalcone, 3′-chloro-2′-hydroxy-4-methoxychalcone, 4′-chloro-2-hydroxy-3-methoxychalcone, 5′-chloro-2′-hydroxy-4′- methoxychalcone, 5′-chloro-2′-hydroxy-4-methoxy-4-methylchalcone, 3′-chloro-2′-hydroxy-4-methylchalcone, 4-chloro-2′-hydroxy-5′-methylchalcone, 5′-chloro-2′-hydroxy-4-methylchalcone, 5′-chloro-2′-hydroxy-4′-methylchalcone, 5′-chloro-2′-hydroxy-4′-methyl-3,4,5-trimethoxychalcone, 5′-chloro-2′-hydroxy-3,4,5-trimethoxychalcone, 4-deoxyphloridzin, 3′,5′-dichloro-2,5-dimethoxy-4′-hydroxychalcone, 2,6-dichloro-2′-hydroxychalcone, 4,5′-dichloro-2′-hydroxychalcone, 3′,5′-dichloro-2′-hydroxy-4-methoxychalcaone, 4,5′-dichloro-2′-hydroxy-4′-methylchalcone, 3′,5′-dichloro-2′-hydroxy-3,4,5-trimethoxychalone, 2,2′-dihydroxychalcone, 3,2′-dihydroxychalcone, 4,2′-dihydroxychalcone, 2′,4′-dihydroxychalcone, 2′,5′-dihydroxychalcone, 2,2′-dihydroxy-4′-6′-dimethoxychalcone, 2′,4′-dihydroxy-2,3-dimethoxychalone, 2′,4′-dihydroxy-3,4-dimethoxychalcone, 2′,6′-dihydroxy-4,4′-dimethoxychalcone, 4,2′-dihydroxy-4′6′-dimethoxychalcone, 2′,6′-dihydroxy-4,4′-dimethoxydihydrochalcone, 2,2′-dihydroxy-3-methoxychalcone, 2,′4′-dihydroxy-2-methoxychalcone, 2′,4′-dihydroxy-4-methoxychalcone, 2′,5′-dihydroxy-4-methoxychalcone, 2′,6′-dihyrdoxy-4′-methoxychalcone, 4,2′-dihydroxy-3-methoxychalcone, 2′,6′-dihydroxy-4-methoxychalcone-4′-O-neohesperidoside, 4,2′-dihydroxy-3-methoxy-5′-methylchalcone, 3,2′-dihydroxy-4,4′,6′-trimethoxychalcone, 4,2′-dihydroxy-3,4′,6′-trimethoxychalcone, 4,′6′-dimethoxy-4-dimethylamino-2′-hydroxychalcone, 2,5-dimethoxy-5′-fluoro-2′-hydroxychalcone, 4′,6′-dimethoxy-4-fluoro-2′-hydroxychalcone, 2,3-dimethoxy-2′-hydroxychalcone, 2,4-dimethoxy-2′-hydroxychalcone, 2,4′-dimethoxy-2′-hydroxychalcone, 2,5-dimethoxy-2′-hydroxychalcone, 2,5′-dimethoxy-2′-hydroxychalcone, 3,4-dimethoxy-2′-hydroxychalcone, 3,4′-dimethoxy-2-hydroxychalchone, 3,4′-dimethoxy-2′-hydroxychalcone, 3′,4′-dimethoxy-2′-hydroxychalcone, 4,4′-dimethoxy-2′-hydroxychalcone, 4′,6′-dimethoxy-2′-hydroxychalcone, 2,4-dimethoxy-2′-hydroxy-5′-methylchalcone, 2,5-dimethoxy-2′-hydroxy-5′-methylchalcone, 3,4-dimethoxy-2′-hydroxy-5′-methylchalcone, 4′,6′-dimethoxy-2′-hydroxy-4-methylchalcone, 4′6′-dimethoxy-2′-hydroxy-3-nitrochalcone, 5′-fluoro-2′-hydroxy-4-methoxychalcone, homobutein, 2-hydroxychalcone, 2′-hyroxychalcone, 4-hydroxychalcone, 4′-hydroxychalcone, 2′-hydroxy-2-methoxychalcone, 2-hydroxy-3-methoxychalcone, 2′-hydroxy-3-methoxychalcone, 2′-hydroxy-4-methoxychalcone, 2′-hydroxy-4′-methoxychalcone, 2′-hydroxy-6′-methoxychalcone, 2′-hydroxy-4-methoxy-5′-methylchalcone, 4′-hydroxy-4-methoxy-2′-methylchalcone, 2′-hydroxy-4-methylchalcone, 2′-hydroxy-5′-methylochalcone, 2′-hydroxy-3,4-methylenedioxychalcone, 2′-hydroxy-5′-methyl-2-methoxychalcone, 4′-hydroxy-2′-methyl-3,4,5-trimethoxychalcone, 2′-hydroxy-2,4,4′,5,6′-pentamethoxychalcone, 2′-hydroxy-2,3,4′,6′-tetramethoxychalcone, 2′-hydroxy-2,4′,5,6′-tetramethoxychalcone, 2′-hydroxy-3,4′,4′,5-tetramethoxychalcone, 2′-hydroxy-3,4,4′,6′-tetramethoxychalcone, 3-hydroxy-2′,4,4′,6′-tetramethoxychalcone, 4-hydroxy-2′,3,4′,6′-tetramethoxychalcone, 4′-hydroxy-2,3′,5,5′-tetramethoxychalcone, 2′-hydroxy-3′,4,5′-trichlorochalcone, 2′-hydroxy-2,3,4′-trimethoxychalcone, 2′-hydroxy-2,3,5′-trimethoxychalcone, 2′-hydroxy-2,4,4′-trimethoxychalcone, 2′-hydroxy-2,4′,5-trimethoxychalcone, 2′-hydroxy-2,4,5′-trimethoxychalcone, 2-hydroxy-2′,4′,6′-trimethoxychalcone, 2′-hydroxy-2,4′,6′-trimethoxychalcone, 2′-hydroxy-3,4,4′-trimethoxychalcone, 2′-hydroxy-2,5,6′-trimethoxychalcone, 2′-hydroxy-3,4,5-trimethoxychalcone, 2′-hydroxy-3,4,5′-trimethoxychalcone, 2′-hydroxy-4,4′,6′-trimethoxychalcone, 4-hydroxy-2′,4′,6′-trimethoxychalcone, marein, 3-methoxy-4,2′,5′-trihydroxychalcone, neohesperidin dihydrochalcone, 3,4,2═,4′,6′-pentahydroxychalcone, 2,2′,4′-trihydroxychalcone, 2,2′,5′-trihydroxychalcone, 2′,3′,4′-trihydroxychalcone, 4,2′,4′-trihydroxychalcone, 4,2′,5′-trihydroxychalcone, and the like), and others (catechin, and the like).

In some embodiments, R₁ in the at least one compound of Formula I may be a flavonoid. In other embodiments, the flavonoid may be chosen from chrysergonic acid, 6-bromochromone-2-carboxylic acid, 6-chloro-7-methylchromone-2-carboxylic acid, chromone-2-carboxylic acid, chromone-3-carboxylic acid, 6-methylchromone-2-carboxylic acid, coumarin-3-carboxylic acid, mercumallylic acid, 5,7-dihydroxy-4-methylcoumarin-3-acetic acid, 7,8-dihydroxy-4-methylcoumarin-3-acetic acid, 7-hydroxycoumarin-4-acetic acid, 7-hydroxycoumarin-3-carboxylic acid, 7-hydroxy-4-methylcoumarin-3-acetic acid, and (7-methoxycoumarin-4-yl)-acetic acid.

The compounds of the present invention comprise from 1 to 900 units of the group of Formula VII:

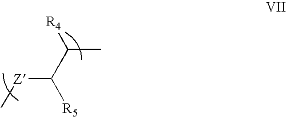

wherein each of R₄, and R₅ is independently chosen from —H, alkyl, substituted alkyl, cycloalkyl, substituted cycloalkyl, aryl, substituted aryl, aralkyl and substituted aralkyl; and

Z′ is chosen from —O—, —N—, —NO—, —NR₄—, —S—, —SO— and —SO₂—; wherein R₄ is defined as above.

Each individual unit of Formula VII may be the same or different from the other units of Formula VII. The compounds of the invention may comprise any combination of individual units of Formula VII (i.e. combinations of polyethylene and/or polypropylene glycol and/or glycerol ethoxylate and/or glycerol propoxylate moieties, where Z′=O). In some embodiments, the compound comprises from 1 to 900 repeats of the same unit of Formula VII.

The compounds of the present invention do not include TPGS compounds with polyethylene glycol chain lengths between 200 and 40,000 Da (i.e., n=4 to 900).

Also provided are compositions comprising at least one compound of Formula I or Formula II and at least one lipophilic compound. In some embodiments, the compositions comprise at least one compound of Formula I and at least one lipophilic compound. In other embodiments, the R₁ group of the at least one compound of Formula I may be a steroid. In some embodiments, the steroid that may be a bile salt. In some embodiments, the steriod may be a cholesterol or a derivative thereof. In other embodiments the steroids may be chosen from cholic acid, deoxy-cholic acid, litho-cholic acid, ursodeoxy-cholic acid, dehydro-cholic acid, cheno-deoxy-cholic acid, glycocholic acid, taurocholic acid, cholesterol, allocholesterol, campesterol, cholanic acid, cholestanol, coprosterol, 7-dehydrocholesterol, dehydroergosterol, 7-dehydrositosterol, desmosterol, lanosterol, α₁-sitosterol, β-sitosterol, γ-sitosterol, stigmastanol, and stigmasterol. In some embodiments, the R₁ group of the at least one compound of Formula I may be a flavonoid. In other embodiments, the flavonoid may be chosen from chrysergonic acid, 6-bromochromone-2-carboxylic acid, 6-chloro-7-methylchromone-2-carboxylic acid, chromone-2-carboxylic acid, chromone-3-carboxylic acid, 6-methylchromone-2-carboxylic acid, coumarin-3-carboxylic acid, mercumallylic acid, 5,7-dihydroxy-4-methylcoumarin-3-acetic acid, 7,8-dihydroxy-4-methylcoumarin-3-acetic acid, 7-hydroxycoumarin-4-acetic acid, 7-hydroxycoumarin-3-carboxylic acid, 7-hydroxy-4-methylcoumarin-3-acetic acid, and (7-methoxycoumarin-4-yl)-acetic acid. In some embodiments, the lipophilic compound may be a pharmaceutical compound. In other embodiments, the lipophilic compound may be chosen from an antibiotic compound, an antifungal compound, an anticancer compound and a cardiovascular drug.

Further provided is a method of inhibiting efflux comprising administering at least one compound of Formula I or Formula II to a subject.

Additionally provided herein is a method of increasing the bioavailability of a lipophilic compound comprising administering at least one composition of the present invention to a subject.

Also provided are methods of increasing the bioavailability of a lipophilic compound comprising administering a compound of Formula I or II to a subject before or at the same time that the lipophilic compound is administered to the subject.

Also provided is a method of formulating a composition comprising providing at least one lipophilic compound, identifying one or more compounds of Formula I or Formula II that will cause a desired degree of efflux administration when administered to a subject, and combining the at least one lipophilic compound and the one or more compounds of Formula I or Formula II to form a composition. In some embodiments, the method of formulating a composition comprises providing at least one lipophilic compound, identifying one or more compounds of the Formula I that will cause a desired degree of efflux administration when administered to a subject and combining the at least one lipophilic compound and the one or more compounds of Formula I to form a composition.

Further provided is a composition comprising at least one compound of Formula I or Formula II and one or more compounds for pharmaceutical use, wherein, upon administration of the composition to a subject, the composition releases the at least one compound of Formula I or Formula II before the composition releases the one or more compounds for pharmaceutical use.

Additional embodiments of the invention are set forth in the description which follows, or may be learned by practice of the invention.

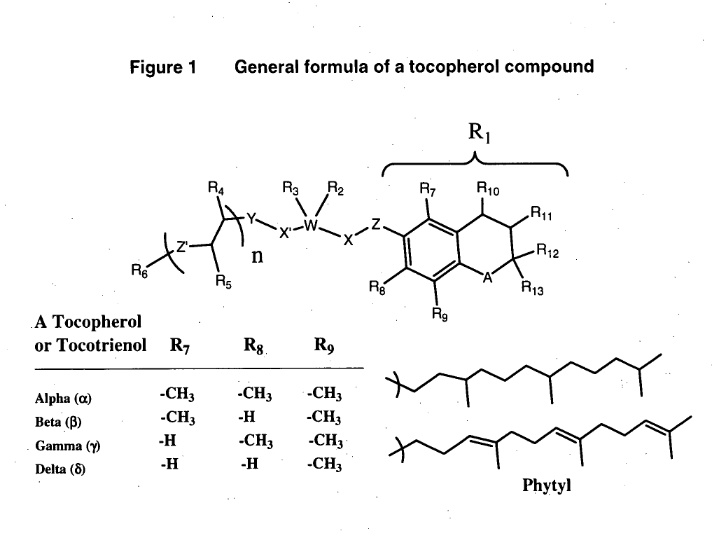

FIG. 1 illustrates the general formula of a compound of the present invention wherein R₁ is a tocopherol (e.g., vitamin E, α-tocopherol). Tocopherol compounds (i.e. tocol, α-tocopherol, β-tocopherol, γ-tocopherol, δ-tocopherol, ε-tocopherol, ζ₁-tocopherol, ζ₂-tocopherol, η-tocopherol, and the like—S. Budavari, editor, The Merck Index (1989), Merck & Co., Inc., Rahway, N.J.) include tocotrienols, which are similar in structure to the tocopherols with the exception of three double bonds in the phytyl side chain.

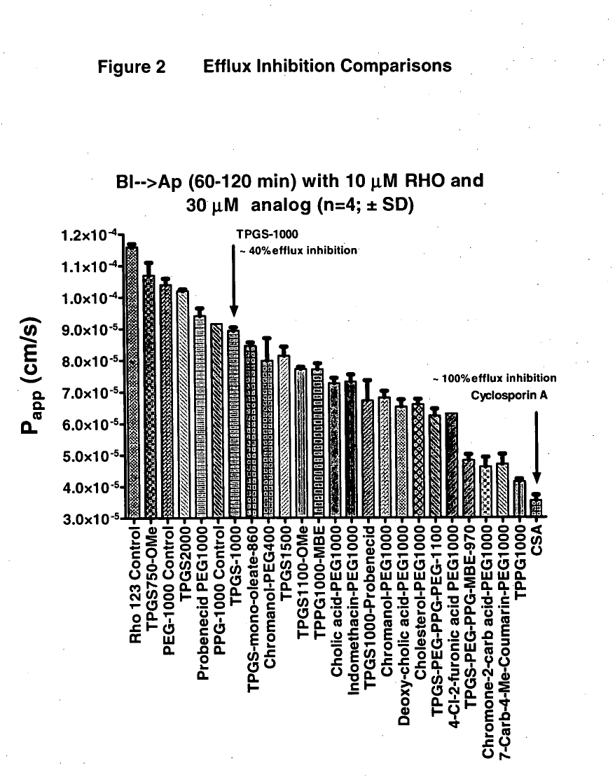

FIG. 2 illustrates in vitro efflux inhibition values for compounds of the present invention.

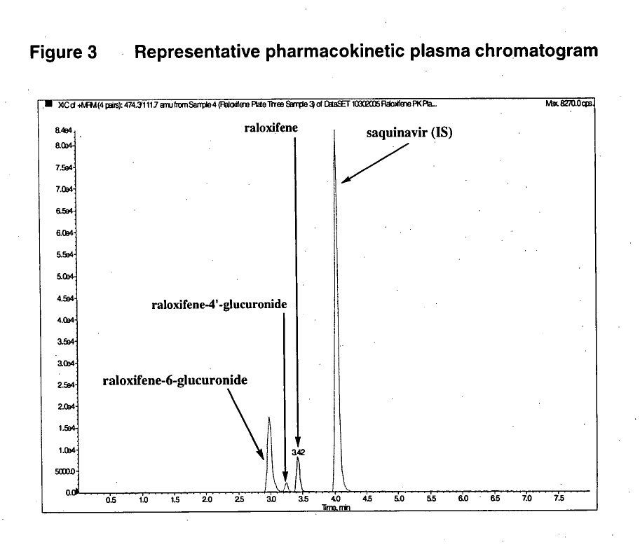

FIG. 3 illustrates a representative pharmacokinetic plasma chromatogram from the analysis of plasma samples by LC/MS/MS.

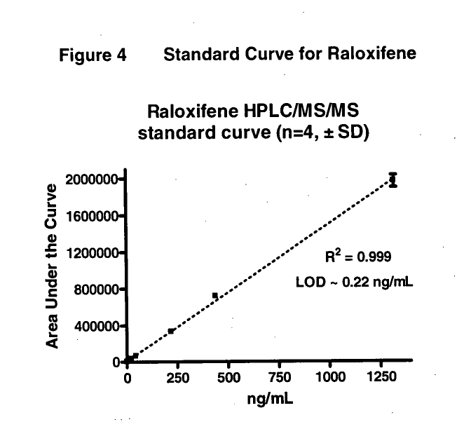

FIG. 4 illustrates the standard curve obtained using plasma samples spiked with the indicated concentrations of raloxifene.

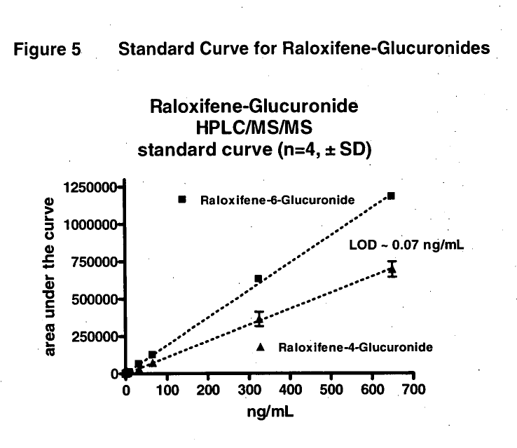

FIG. 5 illustrates the standard curve obtained using plasma samples spiked with the indicated concentrations of raloxifene glucuronides.

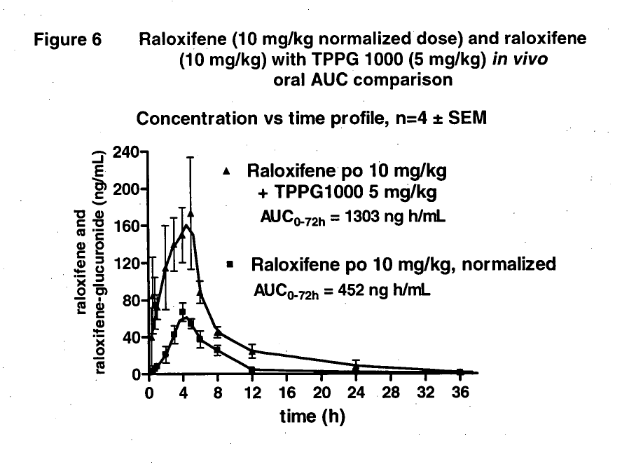

FIG. 6 illustrates the plasma concentration of raloxifene over time after oral dosing of raloxifene alone or raloxifene with TPPG 1000.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, when any variable occurs more than one time in a chemical formula, its definition on each occurrence is independent of its definition at every other occurrence. When the chemical structure and chemical name conflict, the chemical structure is determinative of the identity of the compound. The compounds of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

Compounds of Formula I or Formula II include, but are not limited to optical isomers of compounds of Formula I or Formula II, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula I or Formula II include Z- and E-forms (or cis- and trans-forms) of compounds with double bonds. Where compounds of Formula I or Formula II exist in various tautomeric forms, chemical entities of the present invention include all tautomeric forms of the compound.

Compounds of the present disclosure include, but are not limited to compounds of Formula I or Formula II and all pharmaceutically acceptable forms thereof. Pharmaceutically acceptable forms of the compounds recited herein include pharmaceutically acceptable salts or esters, solvates, crystal forms (including polymorphs and clathrates), chelates, non-covalent complexes, prodrugs, and mixtures thereof. In certain embodiments, the compounds described herein are in the form of pharmaceutically acceptable salts. As used henceforth, the term “compound” encompasses not only the compound itself, but also a pharmaceutically acceptable salt thereof, a solvate thereof, a chelate thereof, a non-covalent complex thereof, a prodrug thereof, and mixtures of any of the foregoing.

As noted above, prodrugs also fall within the scope of chemical entities, for example, ester or amide derivatives of the compounds of Formula I or Formula II. The term “prodrugs” includes any compounds that become compounds of Formula I or Formula II when administered to a patient, e.g., upon metabolic processing of the prodrug. Examples of prodrugs include, but are not limited to, acetate, formate, and benzoate and like derivatives of functional groups (such as alcohol or amine groups) in the compounds of Formula I or Formula II.

As used throughout this application, the term molecular weight, including the abbreviation MW, shall refer, in connection with a single molecule, to the molecular weight of that molecule. With respect to a poly-disperse preparation containing polymer molecules of differing molecular weights, molecular weight shall refer to weight-average molecular weight (M_{w}).

“Alkyl” refers to a saturated or unsaturated, branched, straight-chain or cyclic hydrocarbon group derived by the removal of one hydrogen atom from a single carbon atom of a parent alkane, alkene or alkyne. Typical alkyl groups derived from alkanes include, but are not limited to, methyl, ethyl, propyls such as propan-1-yl, propan-2-yl, and cyclopropan-1-yl, butyls such as butan-1-yl, butan-2-yl, 2-methyl-propan-1-yl, 2-methyl-propan-2-yl, cyclobutan-1-yl, tert-butyl, and the like. Alkyl groups may also contain at least one carbon-carbon double bond. The group may be in either the Z- and E-forms (or cis or trans conformation) about the double bond(s). Typical alkyl groups derived from alkenes include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like. Alkyl groups may also contain at least one carbon-carbon triple bond. Typical alkyl groups derived from alkynes include, but are not limited to, ethynyl; propynyl; butenyl, 2-pentynyl, 3-pentynyl, 2-hexynyl, 3-hexynyl and the like. In certain embodiments, the alkyl group can be a C₁₋₃₀ n-alkyl or C₃₋₄₈ branched alkyl group. In other embodiments, the alkyl group can be a C₁₋₂₂ n-alkyl or C₃₋₄₂ branched alkyl group. In still other embodiments, the alkyl group can be a C₁₋₁₂ n-alkyl or C₃₋₁₂ branched alkyl group. Compounds of the invention containing suitable alkyl groups are capable of inhibiting efflux or increasing the bioavailability of a lipophilic compound.

“Aryl” refers to a monovalent aromatic hydrocarbon group derived by the removal of one hydrogen atom from a single carbon atom of a parent aromatic ring system. Aryl encompasses 5- and 6-membered carbbcyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. For example, aryl includes 5- and 6-membered carbocyclic aromatic rings fused to a 5- to 7-membered heterocycloalkyl ring containing 1 or more heteroatoms chosen from N, O, and S. In certain embodiments, an aryl group can comprise from 6 to 10 carbon atoms. Compounds of the invention containing suitable aryl groups are capable of inhibiting efflux or increasing the bioavailability of a lipophilic compound.

“Arylalkyl” or “aralkyl” refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Arylalkyl moieties include arylalkenyl and arylalkynyl groups. In certain embodiments, an arylalkyl group can be (C₆₋₃₀) arylalkyl, e.g., the alkyl group of the arylalkyl group can be (C₁₋₁₀) and the aryl moiety can be (C₅₋₂₀). Compounds of the invention containing suitable arylalkyl groups are capable of inhibiting efflux or increasing the bioavailability of a lipophilic compound.

“Cycloalkyl” refers to a saturated or unsaturated cyclic alkyl group, including cycloalkanyl and cycloalkenyl groups. Typical cycloalkyl groups include, but are not limited to, groups derived from cyclopropane, cyclobutane, cyclopentane, cyclohexane, and the like. In certain embodiments, the cycloalkyl group can be a C₃₋₈ cycloalkyl or substituted C₃₋₈ cycloalkyl group. Compounds of the invention containing suitable cycloalkyl groups are capable of inhibiting efflux or increasing the bioavailability of a lipophilic compound.

“Halogens” refer to fluorine, chlorine, bromine, or iodine.

“Substituted” refers to a group in which one or more hydrogen atoms are each independently replaced with the same or different substituent(s). Typical substituents include, but are not limited to, —X, —R₃₃, —OH, ═O, —OR₃₃, —SR₃₃, —SH, ═S, —NR₃₃R₃₄, ═NR₃₃, —CX₃, —CF₃ —CN, —NO₂, —S(O)₂R₃₃, —OS(O₂)OH, —OS(O₂)R₃₃, —OP(O)(OR₃₃)(OR₃₄), —(O)R₃₃, —C(S)R₃₃, —C(O)OR₃₃, —C(O)NR₃₃R₃₄, —C(O)OH, —C(S)OR₃₃, —NR₃₅C(O)NR₃₃R₃₄, —NR₃₅C(S)NR₃₃R₃₄, —NR₃₅C(NR₃₃)NR₃₃R₃₄, —C(NR₃₃)NR₃₃R₃₄, —S(O)₂NR₃₃R₃₄, —NR₃₅S(O)₂R₃₃, —NR₃₅C(O)R₃₃, and —S(O)R₃₃ where each X is independently a halogen; each R₃₃ and R₃₄ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, —NR₃₅R₃₆, —(O)R₃₅ or —S(O)₂R₃₅ or optionally R₃₃ and R₃₄ together with the atom to which R₃₃ and R₃₄ are attached form one or more cycloheteroalkyl, substituted cycloheteroalkyl, heteroaryl, or substituted heteroaryl rings; and R₃₅ and R₃₆ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl or substituted heteroarylalkyl, or optionally R₃₅ and R₃₆ together with the nitrogen atom to which R₃₅ and R₃₆ are attached form one or more cycloheteroalkyl, substituted cycloheteroalkyl, heteroaryl, or substituted heteroaryl rings. In certain embodiments, a tertiary amine or aromatic nitrogen may be substituted with on or more oxygen atoms to form the corresponding nitrogen oxide.

The term “solvate” refers to the compound formed by the interaction of a solvent and a compound. Suitable solvates are pharmaceutically acceptable solvates, such as hydrates, including monohydrates and hemi-hydrates.

“Disease” refers to any disease, disorder, condition, symptom, or indication.

The term “drug” or “pharmaceutical” refers to any substance which, when administered to a human or animal under conditions effective to cause absorption to the bloodstream, or into target cells, tissues, or organs, causes a therapeutic or prophylactic effect. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmondics, appetite suppressants, neuroactive substances, neurotransmitter agonists, antagonists, receptor blockers and reuptake modulators, beta-adrenergic blockers, calcium channel blockers, disulfarim and disulfarim-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, and monoamine oxidase inhibitors.

The term “increasing bioavailability” or “increased bioavailability” of a lipophilic compound means that the administration of a compound of the present invention with the lipophilic compound results in an increase in the portion of the dose of the lipophilic compound that reaches one or more targeted systemic fluids, organs, tissues or cells as compared to administration of the lipophilic compound without the compound of the present invention. In vitro and in vivo assays known in the art may be used to assess the relative bioavailability of lipophilic compounds in the presence or absence of compounds of the present invention. For example, in vitro assays employing Caco-2 cells and in vivo animal studies such as those discussed below may be used.

Increased bioavailability can include any mechanism that that has a desired effect on cellular efflux, cellular influx, or clearance. “Clearance” includes any type of elimination of one or more compounds from cells, blood, plasma, tissues or organs (e.g. intestinal clearance, hepatic clearance, renal clearance, and pulmonary clearance each describe elimination of compounds from the blood). Clearance may be described via the observed differences of renal excretion and elimination by all other processes including influx and efflux mechanisms (e.g. gastrointestinal clearance, excretory clearance, biliary clearance and enterohepatic cycling, metabolic clearance). Examples of systemic fluids include, but are not limited to: blood; cerebrospinal fluid; lymph; and any other tissue fluids (including increased amounts in tissues that are bathed by such fluids, such as the brain, tissue of one or more visceral organs, connective tissue, muscle, fat, or one or more tissues in the skin). In some embodiments, the increase is systemic, as in the case of an increase measurable anywhere in the blood. In some embodiments, the increase is more localized, as is the case with some embodiments involving topical administration in which the increase is measured only in areas near the administration. An increase in portion of the dosage that reaches a fluid or tissue measurable by any reliable means is within this definition, including but not limited to increases identified by measuring the total systemic drug concentration over time after administration. In some embodiments, concentrations are determined by measuring the tissue or fluids themselves, or by measuring fractions thereof (for example, without limitation, serum or plasma in the case of blood). In some embodiments, increases for compounds that are excreted metabolized and/or un-metabolized in urine are determined by measuring levels of compounds or metabolites of the compounds in urine and will reflect an increase in systemic concentrations. In some embodiments an increase in compound bioavailability is defined as an increase in the Area Under the Curve (AUC). AUC is an integrated measure of systemic compound concentrations over time in units of mass-time/volume and is measured from the time compound is administered (time zero) to infinity (when no compound(s) remaining in the body can be measured). Information regarding monitoring substances within a subject are known to persons of ordinary skill in the art and may be found in references such as M. Rowland and T. N. Tozer, Clinical Pharmacokinetics Concepts and Applications (third Ed., 1995), Lippincott Willams and Wilkins, Philadelphia.

The term “efflux inhibitor” or “efflux inhibition” refer to a reduction of the transport of a compound in the basolateral to apical (Bl-Ap) direction or a reduction in the ratio of the transport of a compound in the basolateral to apical (Bl-Ap) direction to the transport of the compound in the apical to basolateral (Ap-Bl) direction.

As used throughout this application, the term “lipophilic compounds” shall mean compounds having solubility in water that is in the “sparingly soluble” range, or lower. (Persons of ordinary skill in the art will understand that, for compounds that are “sparingly soluble in water,” the quantity of water needed to dissolve one gram of the compound will be in the range beginning at about 30 mL and ending at about 100 mL. Compounds having solubility lower than “sparingly soluble” in water will require greater volumes of water to dissolve the compounds).

The term “lipophilic compound for pharmaceutical use” refers to a lipophilic compound that is also a compound for pharmaceutical use. Examples of lipophilic compounds for pharmaceutical use include, but are not limited to, itraconazole, astemizole, saquinavir, amprenavir, paclitaxel, docetaxel, doxorubicin, ibuprofen, posaconazole, tacrolimus, danazol, estrogen, lopinavir, tamoxifen, nevirapine, efavirenz, delaviridine, nelfinavir, raloxifene and ritonavir.

“Pharmaceutically acceptable” refers to generally recognized for use in animals, and more particularly in humans.

“Pharmaceutically acceptable salt” refers to a salt of a compound that is pharmaceutically acceptable and that possesses the desired pharmacological activity of the parent compound. Such salts may include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Pharmaceutically acceptable excipient,” “pharmaceutically acceptable carrier,” or “pharmaceutically acceptable adjuvant” refer, respectively, to an excipient, carrier or adjuvant with which at least one compound of the present disclosure is administered. “Pharmaceutically acceptable vehicle” refers to any of a diluent, adjuvant, excipient or carrier with which at least one compound of the present disclosure is administered.

“Stereoisomer” refers to an isomer that differs in the arrangement of the constituent atoms in space. Stereoisomers that are mirror images of each other and optically active are termed “enantiomers” and stereoisomers that are not mirror images of one another and are optically active are termed “diastereoisomers.”

“Subject” includes mammals and humans. The term encompasses cells derived from a subject as well as the organism as a whole.

“Therapeutically effective amount” or “effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying or preventing the onset or reoccurence of the disease or disorder or at least symptoms thereof in a subject which may be exposed to or predisposed to or may have previously suffered from a disease or disorder even though that subject does not yet experience or display symptoms of the disease or disorder.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

Representative compounds of the present invention are disclosed in Table I. The chemical structures of these representative compounds are illustrated in Table II.

TABLE I
Representative Compounds
Compound Description

Chromanol-Succinate-PEG 1000
Chromanol-Succinate-PEG 400
PEG-r-PPG-970-BE-VitE succinate
PEG-b-PPG-b-PEG-1100-VitE succinate
PPG 1000-VitE succinate (TPPG 1000)
BE-PPG-1000-VitE succinate
VitE-succinate-Oleate-860
Ibuprofen-PEG 1000
Indomethacin-PEG 1000
Chromone-2-COOH-PEG 1000
Naproxen-PEG 1000
Probenecid-PEG 1000
Cholesterol-Succinate-PEG 1000
7-carboxymethoxy-4-methyl-coumarin-PEG 1000
5-(4-chlorophenyl)-2-furoic acid-PEG 1000
Cholic acid-PEG 1000
Deoxy-cholic acid-PEG 1000
Probenecid-PEG 1000-Succinate-VitE
Lithocholic acid-PEG 1000
Mono-methyl-ether-PEG 1100-succinate-VitE
PEG 1500-succinate-VitE
Ursodeoxycholic acid-PEG 1000
Dehydrocholic acid-PEG 1000
Chenodeoxycholic acid-PEG 1000
Chromone-3-carboxylic acid-PEG 1000
7-hydroxy-coumarinyl-4-acetic acid-PEG 1000
Tocopheryl-oxy-butyric acid-PEG 1000
Tocopheryl-oxy-acetic acid-PEG 1000
Vit E Succinate-glycerol propoxylate-PEG 1000
Vit E Succinate-glycerol propoxylate-PEG 1500
TPGS 750-OMe
TPGS 2000
TPGS 8000
(R)-(+)-6-hydroxy-2,5,7,8-tetramethyl
chroman-2-carboxylic acid-PEG 1000
TPGS-2000-OMe
VitE-succinate-PEG-PPG-PEG-1900
VitE-succinate-PPG-PEG-MBE-1700
VitE-succinate-PPG 750
VitE-succinate-PPG 2000
VitE-succinate-PPG-PEG-PPG-2000
TPGS 8000 diester
Tri-VitE-succinate-glycerol proproxylate-1500
TPGS 14000
TPGS 20000
TPGS 14000 diester
TPGS 20000 diester
VitE-succinate-glycerol ethoxylate 1000
VitE-succinate-PPG 400
VitE-succinate-PPG 1200
Cholesterol-Succinate-PPG 1000
Stigmasterol-succinate-PEG 1000
gamma-TPGS 1000
gamma-VitE succinate PPG 1000
Cholic acid-PPG 1000

TABLE II
Structures of Representative Compounds

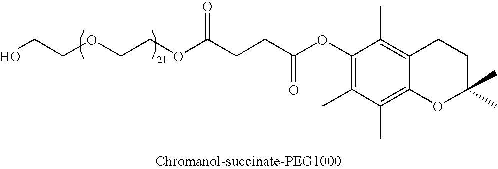

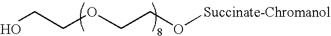

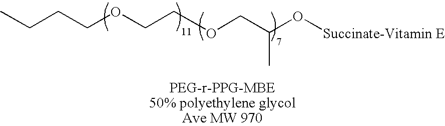

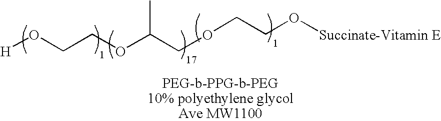

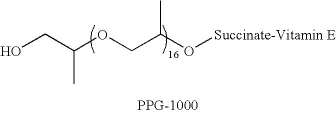

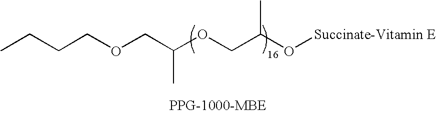

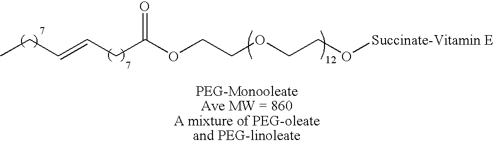

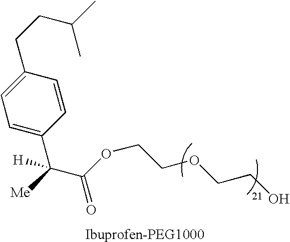

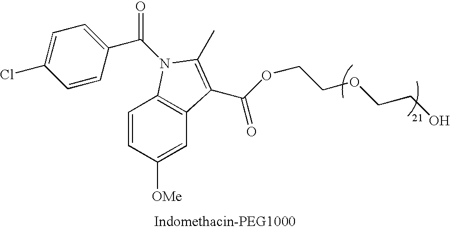

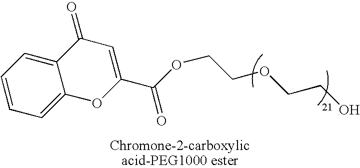

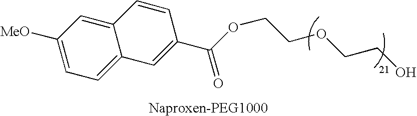

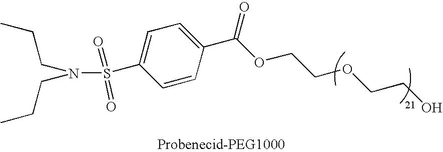

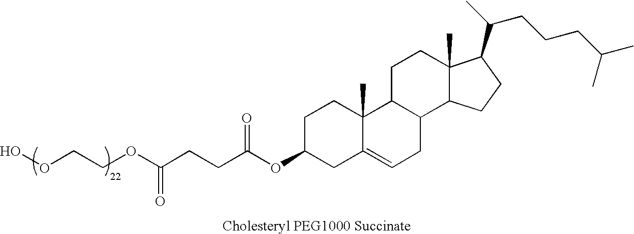

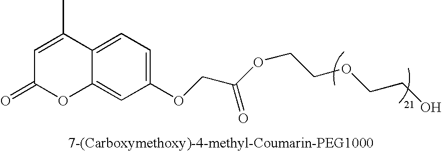

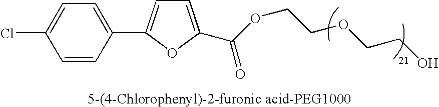

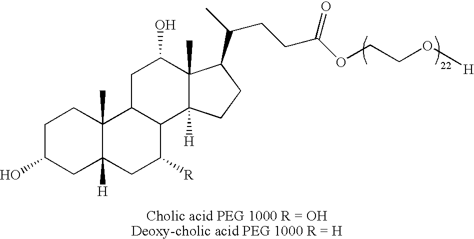

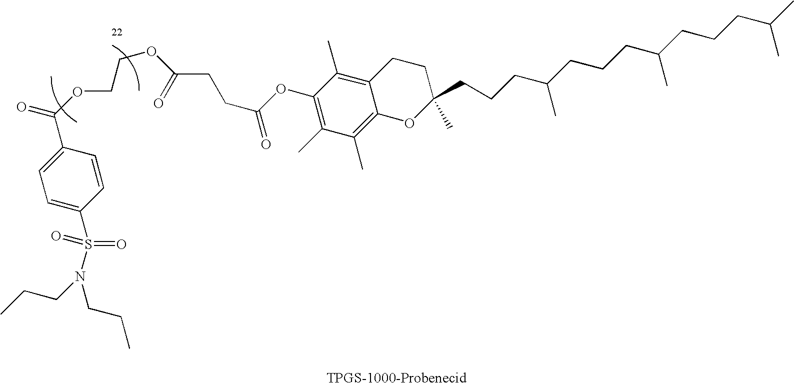

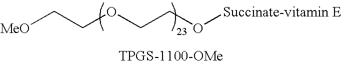

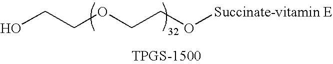

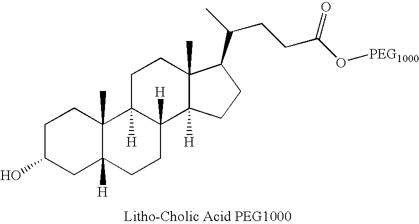

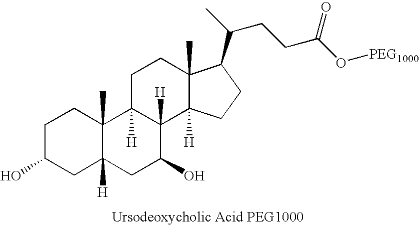

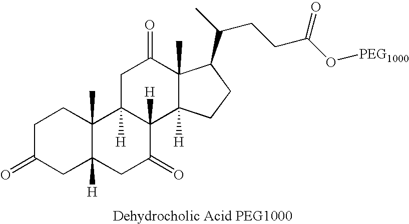

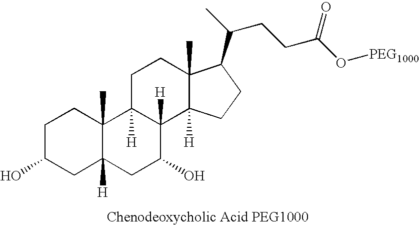

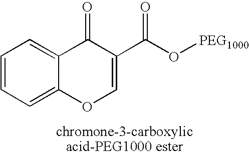

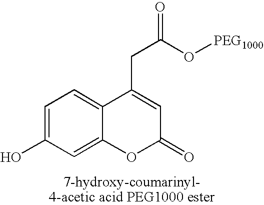

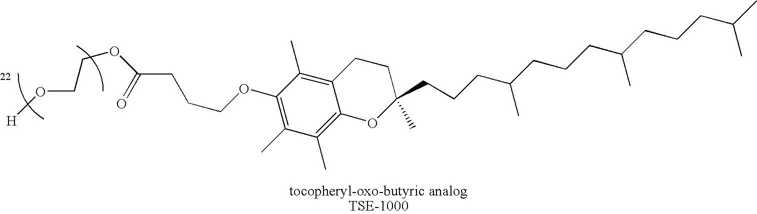

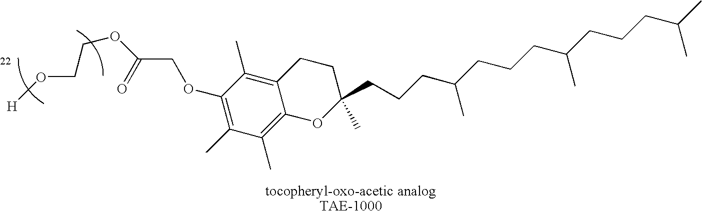

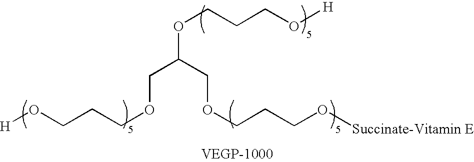

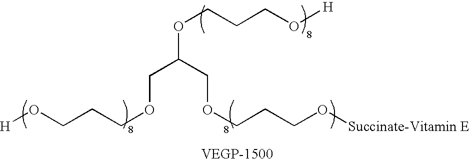

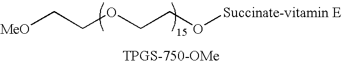

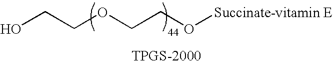

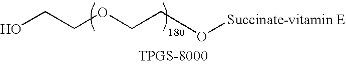

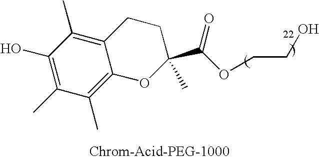

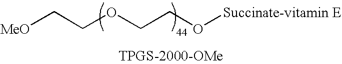

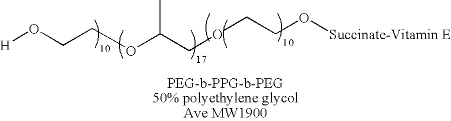

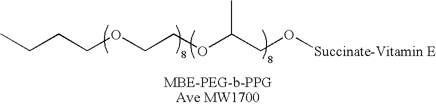

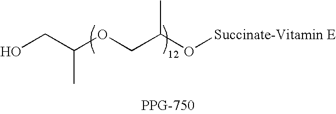

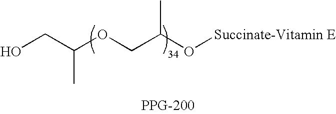

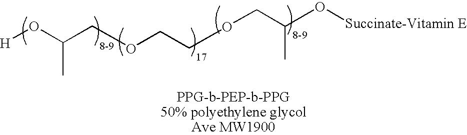

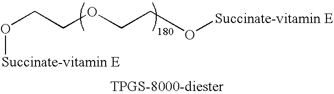

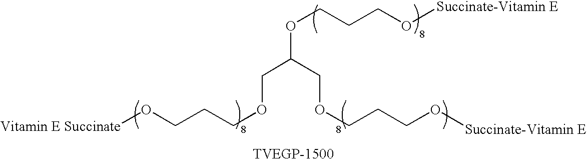

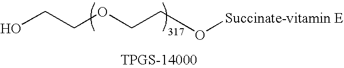

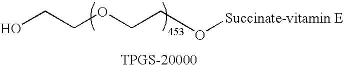

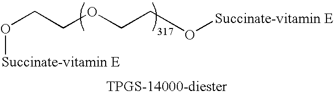

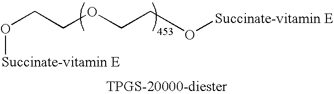

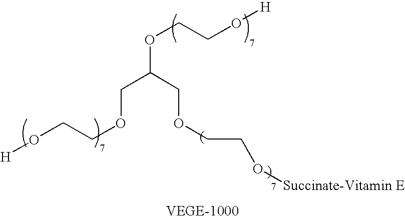

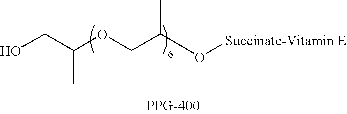

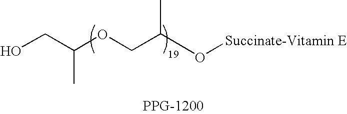

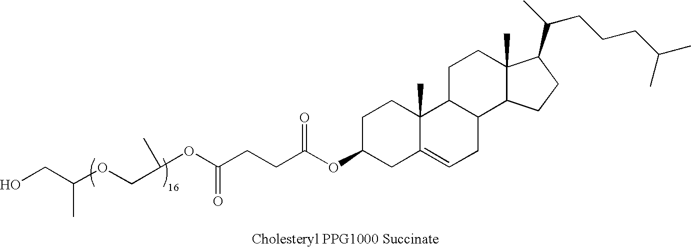

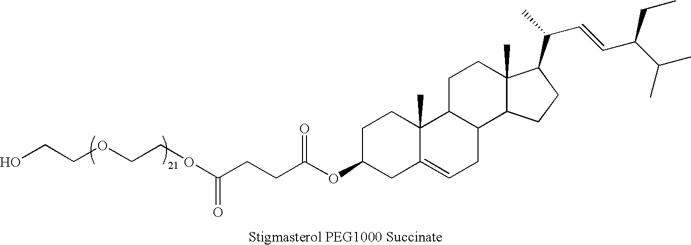

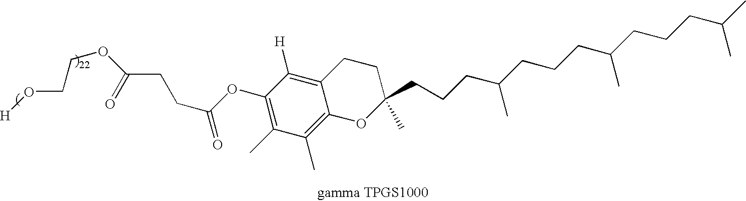

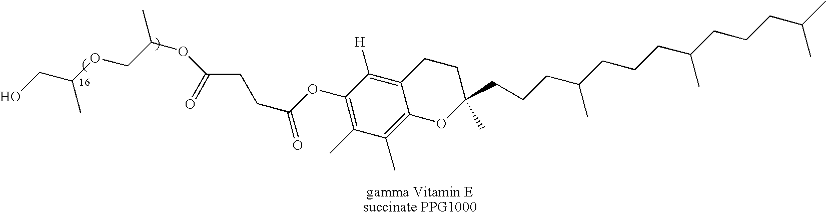

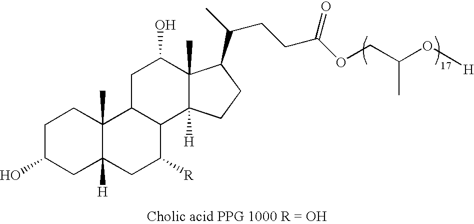

Compounds of the Formula I and Formula II may be made by forming an ester linkage via an activated carboxylic acid and an alcohol, as illustrated by the figure below.

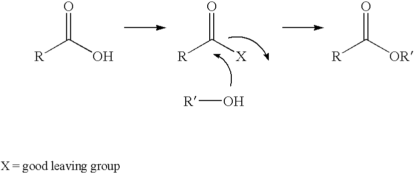

In general, a tocopherol, steroid or flavonoid containing an activated carboxylic acid group is mixed with an alcohol containing molecule of Formula VII to produce a compound of the present invention. In some embodiments, the tocopherol, steroid or flavonoid may contain an alcohol group or be modified to contain an alcohol group and the molecule of Formula VII may contain an activated carboxylic acid group or be modified to contain an activated carboxylic acid group.

Tocopherols, steroids and flavonoids may be modified to contain a carboxylic acid group, such as by the addition of a succinate group, or may contain a carboxylic acid group as part of its unmodified structure. Methods for adding carboxylic acid moieties to compounds are known in the art.

Molecules of Formula VII may be modified to contain an alcohol group or may contain an alcohol group as part of its unmodified structure, such as an ethylene glycol group. Methods for adding alcohol moieties to compounds are known in the art.

Carboxylic acid groups may be activated by a variety of methods known in the art. Examples include the use of an acylating agent (i.e., forming an anhydride), acid halides (using reagents such as thionyl chloride, phosphorous oxychloride, oxalyl chloride, cyanuric chloride, cyanuric fluoride, and the like), using carbodiimide coupling reagents (DCC, EDC, DIC, BEC, CIC, BMC, BDDC, N,N-dicyclopentylcarbodiimide, etc.) in the presence of commonly used activators (DMAP, pyridine, HOBt, HOSu, HOAt, PFpOH), phosphonium reagents (DPPA, MPTA, DECP, BOP-CI), and other known methods.

Molecules of Formula VII are commercially available or may be synthesized by routine methods known in the art. Examples of molecules of Formula VII include, but are not limited to, ethylene glycol, propylene glycol, polyethylene glycol, polypropylene glycol, glycerol ethoxylate, glycerol propoxylate, and the like. In some embodiments, molecules of Formula VII may be monomers; in other embodiments, polymers of a monomeric unit of Formula VII.

Tocopherols, steroids and flavonoids suitable for use in the present invention are known in the art. One of skill in the art will know how to make the tocopherols, steroids or flavonoids or may obtain them from commercial sources.

The invention also includes compositions that contain at least one compound of the present invention. Embodiments of such compositions exist involving all compounds described in this application as well as all combinations of such compounds. In some embodiments, the composition contains one or more lipophilic compounds along with a compound of the present invention. In some embodiments, the lipophilic compound is a lipophilic compound for pharmaceutical use. In some embodiments, the compositions contain a pharmaceutically effective amount of a lipophilic compound for pharmaceutical use. The compound in some embodiments is present above its critical micelle concentration (CMC) and thus increases the solubility of the lipophilic compound in water. In some embodiments, the compound effectively solubilizes the lipophilic compound in water.

Any lipophilic compound known in the art may be used in the compositions of the present invention. Exemplary lipophilic compounds include itraconazole, astemizole, saquinavir, amprenavir, paclitaxel, docetaxel, doxorubicin, ibuprofen, posaconazole, tacrolimus, danazol, estrogen, lopinavir, tamoxifen, nevirapine, efavirenz, delaviridine, nelfinavir, raloxifene, erythromycin, carbamazepine, ketoconazole, indinavir, progesterone, ritonavir, amiodarone, atorvastatin, azithromycin, carvedilol, chlorpromazine, cisapride, ciprofloxacin, cyclosporine, dapsone, diclofenac, diflunisal, flurbiprofen, glipizide, glyburide, griseofulvin, indomethacin, lansoprazole, mebendazole, naproxen, warfarin, terfenadine, talinolol, sirolimus, piroxicam, phentoin, domperidone, and oxaprozin.

In some embodiments, the compositions of the present invention contain one or more additional desirable components or compounds. Any desirable compounds can be used. Examples include, but are not limited to, additional active pharmaceutical ingredients as well as excipients (e.g. cyclodextrins), diluents, and carriers such as fillers and extenders (e.g., starch, sugars, mannitol, and silicic derivatives); binding agents (e.g., carboxymethyl cellulose and other cellulose derivatives, alginates, gelatin, and polyvinyl-pyrrolidone); moisturizing agents (e.g., glycerol); disintegrating agents (e.g., calcium carbonate and sodium bicarbonate); agents for retarding dissolution (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; antioxidants; buffers; bacteriostats; coloring agents; perfuming agents; flavoring agents; lubricants (e.g., talc, calcium and magnesium stearate); solid polyethyl glycols; and mixtures thereof. Examples of carriers include, without limitation, any liquids, liquid crystals, solids or semi-solids, such as water or saline, gels, creams, salves, solvents, diluents, fluid ointment bases, ointments, pastes, implants, liposomes, micelles, giant micelles, and the like, which are suitable for use in the compositions.

It should be understood that the ingredients particularly mentioned above are merely examples and that some embodiments of formulations comprising the compositions of the present invention include other suitable components and agents. The compositions of the invention may be used for, among other things, pharmaceutical and cosmetic purposes and may be formulated with different ingredients according to the desired use.

Compounds of the present invention, lipophilic compounds and any additional components may be combined and formulated in any manner known in the pharmaceutical field.

In some embodiments, the aqueous solubility of a lipophilic compound, when combined with a compound of the present invention, may be greater than the aqueous solubility of the lipophilic compound in the absence of the compound of the present invention. Methods of determining the aqueous solubility of a compound or composition are well known to those of skill in the art.

The compounds or compositions of the invention may be administered to a subject to inhibit efflux. Efflux inhibition may be determined using standard assays such as the Caco-2 cell assay disclosed below. One of skill in the art will recognize that standard assays may also be used to predict efflux inhibition for a compound or composition of the present invention to be administered to a subject such as a human.

Percentage of efflux inhibition may be determined by comparing the amount of a compound (such as Rhodamine 123) transported across a Caco-2 cell monolayer in the presence or absence of a compound of the present invention, as described below. Percentage of efflux inhibition values may be calculated by determining the ratio (efflux ratio) of Rhodamine 123 permeability in the basolateral to apical (Bl-Ap) direction to Rhodamine 123 permeability in the apical to basolateral (Ap-Bl) direction in the presence or absence of a compound of the present invention. In some embodiments, the compounds or compositions of the invention may inhibit efflux by 1-10%; in other embodiments, by 10-20%, 20-30%, 30-40%, 40-50%, 50-60%, 60-70%, 70-80%, 80-90% or 90-100%. In certain embodiments, the compounds or compositions of the invention may inhibit efflux by greater than 75%; in other embodiments, by greater than 85%; in still other embodiments, by greater than 95%.

The compounds and compositions of the present invention may also be used to increase the bioavailability of a lipophilic compound when a compound of the present invention and the lipophilic compound are coadministered. In some embodiments, the compound of the present invention and the lipophilic compound may be administered at the same time. This may be accomplished, for example, by administering them together as separate compounds or as one composition. In other embodiments, the compound of the present invention may be administered before the lipophilic compound. One of skill in the art may determine an increase in the bioavailability of a lipophilic compound using assays standard in the art. For example, the plasma or tissue concentration of a lipophilic compound in an animal may be determined after administration of the lipophilic compound alone or after administration of the lipophilic compound in combination with a compound of the present invention. An example of a suitable animal assay for determining increased bioavailability is disclosed below.

Increases in bioavailability may be determined by measuring the plasma concentration of a lipophilic compound in an animal after dosing with a lipophilic compound in the presence or absence of a compound of the present invention. AUC values may be determined and compared as described in the example below. In some embodiments, the compounds or compositions of the invention may increase the bioavailability of a lipophilic compound by a factor of 0.1 to 10 in comparison to the lipophilic compound alone. In certain embodiments, the increase may be by a factor of 0.1 to 1, 1 to 2, 2 to 3, 3 to 4, 4 to 5, 5 to 6, 6 to 7, 7 to 8, 8 to 9 or 9 to 10.

The compounds or compositions of the invention may be used in any amount effective for efflux inhibition or to increase the bioavailability of a lipophilic compound. One of skill in the art will recognize that effective amounts may vary depending upon, among other variables, the compound of the present invention utilized, the nature of the lipophilic compound, any additional components present in the composition, the size of the patient, the dosage form, the route of administration, and the like. The effective amount of a compound or composition may be routinely determined by one of skill in the art using standard assays such as the in vitro and in vivo assays disclosed herein. For example, animal studies may be used to determine the range of effective amounts and these data may be extrapolated to determine an effective amount for administration to a human.

The effective amount of compounds or compositions of the invention may range from about 0.1 to 100 milligrams (mg) per kilogram (kg) of subject weight. In certain embodiments, the compounds or compositions of the invention are administered at from about 0.1 mg/kg to 2 mg/kg or from about 2 mg/kg to 5 mg/kg; in other embodiments, from about 5 mg/kg to 10 mg/kg, from about 10 mg/kg to 20 mg/kg, from about 20 mg/kg to 30 mg/kg, from about 30 mg/kg to 40 mg/kg, from about 40 mg/kg to 50 mg/kg, from about 50 mg/kg to 75 mg/kg or from about 75 mg/kg to 100 mg/kg.

Compositions of the invention containing a pharmaceutical compound may be administered to a subject to treat or prevent a disease or disorder treatable by the pharmaceutical compound. Administration of compositions of the invention containing a pharmaceutical compound may increase the amount of the pharmaceutical compound in the plasma or tissue of a subject. One of skill in the art will recognize that the amount of pharmaceutical compound present in the composition may have to be altered accordingly to provide the desired effective amount of the pharmaceutical compound.

In some embodiments, the compounds and compositions of the present invention may be administered to a subject. Suitable subjects include a cell, population of cells, tissue or organism. In certain embodiments, the subject is a mammal such as a human. The compounds may be administered in vitro or in vivo.

In some embodiments, the compounds or compositions of the present invention are administered to persons or animals to provide substances in any dose range that will produce desired physiological or pharmacological results. Dosage will depend upon the substance or substances administered, the therapeutic endpoint desired, the desired effective concentration at the site of action or in a body fluid, and the type of administration. Information regarding appropriate doses of substances are known to persons of ordinary skill in the art and may be found in references such as L. S. Goodman and A. Gilman, eds, The Pharmacological Basis of Therapeutics, Macmillan Publishing, New York, and Katzung, Basic & Clinical Pharmacology, Appleton & Lang, Norwalk, Conn., (6.sup.th Ed. 1995).

The compositions can be administered in any form by any means. Examples of forms of administration include, but are not limited to, injections, solutions, creams, gels, implants, ointments, emulsions, suspensions, microspheres, powders, particles, microparticles, nanoparticles, liposomes, pastes, patches, capsules, suppositories, tablets, transdermal delivery devices, sprays, suppositories, aerosols, or other means familiar to one of ordinary skill in the art. In some embodiments, the compositions can be combined with other components. Examples include, but are not limited to, coatings, depots, matrices for time release and osmotic pump components.

Examples of methods of administration include, but are not limited to, oral administration (e.g., ingestion, buccal or sublingual administration), anal or rectal administration, topical application, aerosol application, inhalation, intraperitoneal administration, intravenous administration, transdermal administration, intradermal administration, subdermal administration, intramuscular administration, intrauterine administration, vaginal administration, administration into a body cavity, surgical administration (for example, at the location of a tumor or internal injury), administration into the lumen or parenchyma of an organ, and parenteral administration.

The invention further includes any method of admixture or coadministration, including the above methods, in which the method further includes the step of identifying a desired degree (or lack thereof) of efflux inhibition on the part of the at least one compound of Formula I or Formula II. In some embodiments, the method includes selecting from among two or more compounds of Formula I or Formula II that are compounds for use as an efflux inhibitor to identify the desired level of efflux inhibition. In some embodiments, the method includes selecting from among two or more compounds of Formula I or Formula II that are compounds not for use as an efflux inhibitor to identify the desired level of efflux inhibition. In some embodiments, the method includes selecting from among two or more compounds of Formula I or Formula II that are compounds for use as an efflux inhibitor as well as two or more compounds of Formula I or Formula II that are compounds not for use as an efflux inhibitor to identify the desired level of efflux inhibition. In some embodiments, the method includes selecting a mixture or other combination of a plurality of compounds of Formula I or Formula II to obtain the desired degree of efflux inhibition. Through this method, manipulation of the number and identity of the compounds of Formula I or Formula II selected allows fine control of the degree of efflux inhibition. The selected compounds may then be administered to a subject or formulated with a lipophilic compound and the resulting composition may be administered to a subject.

The invention further includes packages, vessels, or any other type of container that contains either a compound of the present invention or any composition comprising a compound of the present invention. The package, vessel or container contains, is labeled with, or is otherwise accompanied by instructions to use the compound or composition to enhance or increase solubility of one or more lipophilic compounds in water and indicates in any manner that the compound or composition does not inhibit effect on efflux, has a diminished, limited, or insignificant inhibitory effect on efflux, or otherwise provides some indication regarding a lack of efflux inhibition or a reduced degree of efflux inhibition (for example, identifying that the efflux inhibition is no greater than a certain level).

The following are examples of methods that can be used to produce intermediates to and compounds of the present invention.

The structures of representative compounds of the present invention synthesized by the method below are illustrated in Table II above. A general experimental example is summarized as follows: vitamin E succinate (3.25 g, 6.12 mmol) or other starting material (i.e. chromanol succinate, gamma-vitamin E succinate, ibuprofen, naproxen, steroid, flavonoid, etc) was dissolved in dichloromethane (20 mL) and 1.1 equivalents of the corresponding polyethylene glycol, polypropylene glycol, glycerol ethoxylate, glycerol propoxylate, etc. added and stirred at room temperature. DMAP (0.1 equivalents)—in the case of steroids, in place of DMAP, pyridine was added until materials were dissolved—and DCC (1.1 equivalents) were added sequentially. The reaction vessel was capped and stirred overnight. The reaction mixture was filtered through a Buchner funnel, and the filtrate concentrated under reduced pressure to afford crude product(s). The compounds were constructed by forming an ester linkage via an activated carboxylic acid and an alcohol; a well known chemical modification. Formation of the activated carboxylic acid may be accomplished by a variety of known methods; for example, via an acylating agent (i.e., forming an anhydride), acid halides (using reagents such as thionyl chloride, phosphorous oxychloride, oxalyl chloride, cyanuric chloride, cyanuric fluoride, and the like), using carbodiimide coupling reagents (DCC, EDC, DIC, BEC, CIC, BMC, BDDC, N,N-dicyclopentylcarbodiimide, etc.) in the presence of commonly used activators (DMAP, pyridine, HOBt, HOSu, HOAt, PFpOH), phosphonium reagents (DPPA, MPTA, DECP, BOP-Cl), and other published methods. The compounds summarized in Table I were prepared using thionyl chloride and/or via DCC reaction methodology.

Reaction products were purified via preparative HPLC (Varian Dynamax Microsorb C8 column, 250×41.4 mm i.d., 8 μ particles, 60 Å pore) using mobile phases: A, 70/30 [50/50 isopropyl alcohol (IPA)/acetonitrile (ACN)]/0.0025M NH₄OAc; B, 50/50 IPA/ACN; and C, 100% IPA with general step-gradient conditions of A for 24 min, B for 6 min and C for 12 min at a flow rate of ˜65 mL/min.

The following are examples of in vitro and in vivo assays that can be used to evaluate the efflux inhibition or bioavailability properties of compounds and compositions of the present invention.

In Vitro Methodology—Caco-2 Cell Culture and Handling

Caco-2 cells, clone C2BBe1, were purchased from American Type Culture Collection (ATCC; Manassas, Va.). Cells from passage 49-55 with polyethylene terephthalate (PET) membranes (BD Falcon™ HTS Multiwell, 24-well, 1.0 μm pore size, 0.31 cm² growth area and 6.5 mm diameter) were used. Cells were seeded at a density of ˜60,000 cells/cm and grown at ˜37° C. in a controlled atmosphere of ˜5% CO₂ with a relative humidity of ˜85%. The culture medium consisted of DMEM supplemented with 10% FBS, 1% non-essential amino acids, 100 μg/mL streptomycin and 100 U/mL penicillin. Transepithelial electrical resistance (TEER) was measured with an automated REMS (method two) electrical volt-ohm meter EVOM (World Precision Instruments; Sarasota, Fla.). Only monolayers with a TEER>350 Ω*cm², with background subtracted, were used for transport studies.

Transport screening assay—Rhodamine 123 (RHO) transport was assessed in the secretory, basolateral to apical (Bl-Ap) direction in the presence of the indicated compound (30 μM), prepared in HBSS solution and present on both the apical and basolateral sides. Prior to RHO transport experiments, the monolayers were pre-incubated for approximately one hour with the corresponding compound, both at pH 7.4. Subsequently, at t=0 min, a solution of RHO (10 μM) in buffer solution (pH ˜7.4) was added to donor compartment (basolateral side) and pure buffer solution (pH ˜6.5) added to the receiver compartment (apical side). The method did not utilize plate shaking. Samples were collected after 60, 120, and 180 min from the receiver compartment. Samples were taken by complete replacement of the receiver volume with fresh buffer solution (˜37° C.) containing excipient. RHO was quantified using LC/MS/MS.

Mass Spectrometry—Caco-2 Experiments: An Applied Biosystems Sciex 4000-QTrap® (Applied Biosystems; Foster City, Calif.) equipped with a Shimadzu HPLC (Shimadzu Scientific Instruments, Inc.; Columbia, Md.), a PEAK Scientific API Systems gas generator (PEAK Scientific; Bedford, Mass.) and a Leap auto-sampler (LEAP Technologies; Carrboro, N.C.) was used. An Agilent Technologies, Zorbax extended-C18 50×4.6 mm, 5 micron column was used at 40° C. with a flow-rate of 0.4 mL/min. The mobile phase consisted of A: 10 mM ammonium acetate, 0.1% formic acid in water, and B: 50:50 acetonitrile:methanol. HPLC method used a 6.5 min run time; started at 5% B, ramped up to 95% B at 3 min and held until 4.5 min, ramped back down to 5% B at 5 min and held until end of run. Multiple reaction monitoring (MRM) channel in positive ion electrospray (+ESI) mode was used: rhodamine 123, 345.2→285.2 m/z.

Apparent Permeability and Statistical Analysis

Flux was determined using receiver compartment RHO steady-state appearance rates (ΔQ/Δdt). Pₐₚₚ across Caco-2 monolayers was calculated via:

$${P}_{app} = \frac{Δ Q}{Δ t \cdot A \cdot {C}_{0}}$$

Pₐₚₚ is apparent permeability coefficient [cm/s], ΔQ/ΔT is permeability rate [μg/s; pmol/s], C₀ is initial concentration in donor chamber [μg/mL; pmol/cm³], and A is membrane surface area [cm²]. Pₐₚₚ Bl-Ap is expressed as means±standard deviation (SD).

As illustrated by the data summarized in FIG. 2, the Caco-2 protocol set forth may be used to test and rank order the ability of the compounds to influence in vitro efflux. FIG. 2 shows that TPGS 1000 altered RHO in vitro efflux by ˜40% and a pharmaceutical gold-standard, cyclosporine A (CSA), by ˜100%; complete in vitro efflux inhibition. These in vitro data illustrate that compounds with far more potent efflux properties, relative to TPGS 1000, were prepared. For example, four of the in vitro tested compounds inhibited RHO efflux by ≧80%.

In Vivo Experiments—Animals and Dose Administration

Male, Wistar-Hannover rats (257-313 g) were used; animals were cannulated in the jugular vein for blood sampling. Animals were individually housed at 18-26° C. with 30-70% humidity. Animals were allowed free movement and access to water with 12 h dark and light cycles. Dosing was 2-4 h after the beginning of a light cycle and dosing time across each group was consistent. All animals had access to water, ad libitum, and were fasted 8 h prior to dosing; food was returned 5 h post-dose. Animals were dosed using a 1 mL syringe. Group 1, the intravenous group (raloxifene, 2.5 mg/kg), was dosed using an infusion rate of ˜0.3 mL per minute. Groups 2 (12 mg/kg raloxifene) and 3 (raloxifene 10 mg/kg; TPPG 1000 5 mg/kg) were dosed as capsules; following the capsule dosing, 0.5 mL of water was administered to facilitate capsule movement to the stomach.

Sampling—using an AccuSampler®, blood samples (˜120 μL) were collected from 4 animals/group/time point from a catheter inserted into the jugular vein. Blood samples were taken and removed blood volume was replaced by an equivalent volume of intraperitoneal saline after each draw. Blood samples were centrifuged and plasma transferred into a designated well of a 96-well plate. Samples were kept frozen (−80±10° C.) until sample preparation and HPLC/MS/MS analysis.

Raloxifene HCl was purchased from Sigma-Aldrich (St Louis, Mo.). Saquinavir base (Lot # 25449) was purchased from Apin Chemicals Ltd (Abingdon, Oxon, UK). Raloxifene 6-glucuronide (II) (Lot # 19-WG-9-1) and raloxifene 4′-glucuronide (III) (Lot # 18-WG-171-1) were purchased from Toronto Research Chemicals, Inc. (NorthYork, Ontario Canada). Wistar-Hannover plasma (potassium EDTA) was obtained from Bioreclamation Inc. (Hicksville, N.Y.).

Internal standard (IS) solution was prepared in a 500 mL volumetric flask containing 1:1 v/v acetonitrile:methanol and ˜0.04 μM of saquinavir base. Individually, the 96-well plates were removed from the freezer (−80±10° C.) and allowed to warm to ambient temperature (˜45-50 min). The in vivo plasma samples, either 25 or 50 μL, were transferred into separate 1.5 mL micro-centrifuge tubes. Total plasma volume was brought to ˜100 μL by adding male Wistar-Hannover plasma (potassium EDTA). Subsequently, 200 μL of IS solution was added, capped, mixed (˜5 sec) and centrifuged at 13,200 rpm for 10 mm using an Eppendorf minispin centrifuge. The supernatant (˜250 μL) was transferred into individual wells of a 96-well plate. The 96-well plate was sealed and centrifuged at 3000 rpm for 10 min at 10° C. (Labofuge 400R Centrifuge). The 96-well plate was then placed into the auto-sampler cool-stack (˜6° C.) and analyzed via LC/MS/MS. Raloxifene, and raloxifene glucuronide standard curves and quality control (QC's) samples were prepared in an analogous way as described above. A representative pharmacokinetic plasma chromatogram contained raloxifene, saquinavir (IS), and glucuronides II and III (FIG. 3). The standard curves for raloxifene and glucuronides via spiked plasma are presented in FIG. 4 and FIG. 5, respectively. The limit of detection (LOD) for raloxifene was ˜0.2 ng/mL while the glucuronides were ˜0.1 ng/mL. The oral dose (normalized to 10 mg/kg) plasma concentration-time data for raloxifene from group two (oral raloxifene, 10 mg/kg) and group three (Raloxifene, 10 mg/kg; TPPG 1000, 5 mg/kg) is summarized in FIG. 6. A visual inspection reveals that the Cₘₐₓ for group two was ˜50-60 ng/mL with a Tₘₐₓ of ˜4 h; whereas, group three had a Cₘₐₓ ˜150-160 ng/mL with a Tₘₐₓ of ˜3-5 h. These data illustrate that the presence of TPPG 1000 in an oral dose increased the overall amount of raloxifene absorbed; the overall AUC_{0-72 h} increased from 452 ng h/mL to 1303 ng h/mL); dose normalized bioavailability increased from 3.6±0.8% to 10.4±2.8%.

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

What is claimed is:

1. At least one compound of Formula I: 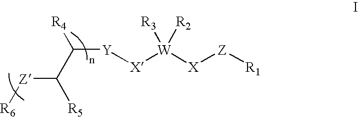
a pharmaceutically acceptable salt or ester thereof, a solvate thereof, a chelate thereof, a non-covalent complex thereof, a prodrug thereof, and mixtures of any of the foregoing, wherein:
n is a number from 1 to 900, wherein the individual units may be the same or different;
W is chosen from alkyl, substituted alkyl, cycloalkyl, substituted cycloalkyl, aryl, substituted aryl, aralkyl and substituted aralkyl;
each of R₂, R₃, R₄ and R₅ is independently chosen from —H, alkyl, substituted alkyl, cycloalkyl, substituted cycloalkyl, aryl, substituted aryl, aralkyl and substituted aralkyl;
Z′ is chosen from —O—, —N—, —NO—, —NR₄—, —S—, —SO— and —SO₂—, wherein R₄ is defined as above;
each of X, X′, Y and Z is independently chosen from —CR₄R₅—, —NH—, —NR₄—, —NO—, —O—, —NOR₄—, —S—, —SO—, —SO₂—, wherein R₄ and R₅ are defined as above;
R₁ is chosen from a tocopherol, a steroid and a flavonoid; and
R₆ is chosen from any R₁, alkyl, substituted alkyl, cycloalkyl, substituted cycloalkyl, aryl, substituted aryl, aralkyl and substituted aralkyl.

2. The at least one compound of claim 1, wherein R₁ is a tocopherol of Formula IV: 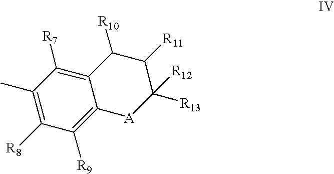
wherein:
each of R₇, R₈, R₉, R₁₀, R₁₁, R₁₂ and R₁₃ is independently chosen from —H, alkyl, substituted alkyl, cycloalkyl, substituted cycloalkyl, aryl, substituted aryl, aralkyl and substituted aralkyl; and
A is chosen from —CR₄R₅—, —NH—, —NR₄—, —O—, —NO—, —NOR₄—, —S—, —SO— and —SO₂—, wherein each of R₄ and R₅ is independently chosen from —H, alkyl, substituted alkyl, cycloalkyl, substituted cycloalkyl, aryl, substituted aryl, aralkyl and substituted aralkyl.

3. The at least one compound of claim 2, wherein the tocopherol is chosen from tocol, α-tocopherol, β-tocopherol, γ-tocopherol, δ-tocopherol, ε-tocopherol, ζ₁-tocopherol, ζ₂-tocopherol, and η-tocopherol.

4. The at least one compound of claim 1, wherein R₁ is a steroid.

5. The at least one compound of claim 4, wherein the steroid is a bile salt.

6. The at least one compound of claim 4, wherein the steroid is cholesterol or a derivative thereof.

7. The at least one compound of claim 4, wherein the steroid is chosen from cholic acid, deoxy-cholic acid, litho-cholic acid, ursodeoxy-cholic acid, dehydro-cholic acid, cheno-deoxy-cholic acid, glycocholic acid, taurocholic acid, cholesterol, allocholesterol, campesterol, cholanic acid, cholestanol, coprosterol, 7-dehydrocholesterol, dehydroergosterol, 7-dehydrositosterol, desmosterol, lanosterol, α₁-sitosterol, β-sitosterol, γ-sitosterol, stigmastanol, and stigmasterol.

8. The at least one compound of claim 1, wherein R₁ is a flavonoid.

9. The at least one compound of claim 8, wherein the flavonoid is chosen from chrysergonic acid, 6-bromochromone-2-carboxylic acid, 6-chloro-7-methylchromone-2-carboxylic acid, chromone-2-carboxylic acid, chromone-3-carboxylic acid, 6-methylchromone-2-carboxylic acid, coumarin-3-carboxylic acid, mercumallylic acid, 5,7-dihydroxy-4-methylcoumarin-3-acetic acid, 7,8-dihydroxy-4-methylcoumarin-3-acetic acid, 7-hydroxycoumarin-4-acetic acid, 7-hydroxycoumarin-3-carboxylic acid, 7-hydroxy-4-methylcoumarin-3-acetic acid, and (7-methoxycoumarin-4-yl)-acetic acid.

10. A composition comprising:
(a) at least one compound according to claim 1; and
(b) at least one lipophilic compound.

11. The composition of claim 10, wherein the R₁ group of the at least one compound is a steroid.

12. The composition of claim 11, wherein the steroid is a bile salt.

13. The composition of claim 11, wherein the steroid is cholesterol or a derivative thereof.

14. The composition of claim 11, wherein the steroid is chosen from cholic acid, deoxy-cholic acid, litho-cholic acid, ursodeoxy-cholic acid, dehydro-cholic acid, cheno-deoxy-cholic acid, glycocholic acid, taurocholic acid, cholesterol, allocholesterol, campesterol, cholanic acid, cholestanol, coprosterol, 7-dehydrocholesterol, dehydroergosterol, 7-dehydrositosterol, desmosterol, lanosterol, α₁-sitosterol, β-sitosterol, γ-sitosterol, stigmastanol, and stigmasterol.

15. The composition of claim 10, wherein the R₁ group of the at least one compound is a flavonoid.

16. The composition of claim 15, wherein the flavonoid is chosen from chrysergonic acid, 6-bromochromone-2-carboxylic acid, 6-chloro-7-methylchromone-2-carboxylic acid, chromone-2-carboxylic acid, chromone-3-carboxylic acid, 6-methylchromone-2-carboxylic acid, coumarin-3-carboxylic acid, mercumallylic acid, 5,7-dihydroxy-4-methylcoumarin-3-acetic acid, 7,8-dihydroxy-4-methylcoumarin-3-acetic acid, 7-hydroxycoumarin-4-acetic acid, 7-hydroxycoumarin-3-carboxylic acid, 7-hydroxy-4-methylcoumarin-3-acetic acid, and (7-methoxycoumarin-4-yl)-acetic acid.

17. The composition of claim 10, wherein the lipophilic compound is a pharmaceutical compound.

18. The composition of claim 17, wherein the lipophilic compound is chosen from an antibiotic compound, an antifungal compound, an anticancer compound and a cardiovascular drug.

19. A method of formulating a composition comprising:
(a) providing at least one lipophilic compound;
(b) identifying one or more compounds of the Formula I: 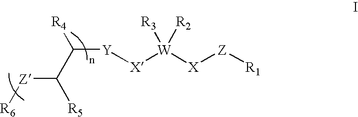
a pharmaceutically acceptable salt or ester thereof, a solvate thereof, a chelate thereof, a non-covalent complex thereof, a prodrug thereof, and mixtures of any of the foregoing, wherein:
n is a number from 1 to 900, wherein the individual units may be the same or different;
W is chosen from alkyl, substituted alkyl, cycloalkyl, substituted cycloalkyl, aryl, substituted aryl, aralkyl and substituted aralkyl;
each of R₂, R₃, R₄ and R₅ is independently chosen from —H, alkyl, substituted alkyl, cycloalkyl, substituted cycloalkyl, aryl, substituted aryl, aralkyl and substituted aralkyl;
Z′ is chosen from —O—, —N—, —NO—, —NR₄—, —S—, —SO— and —SO₂—, wherein R₄ is defined as above;
each of X, X′, Y and Z is independently chosen from —CR₄R₅—, —NH—, —NR₄—, —NO—, —O—, —NOR₄—, —S—, —SO—, —SO₂—, wherein R₄ and R₅ are defined as above;
R₁ is chosen from a tocopherol, a steroid and a flavonoid; and R₆ is chosen from any R₁, alkyl, substituted alkyl, cycloalkyl, substituted cycloalkyl, aryl, substituted aryl, aralkyl and substituted aralkyl that will cause a desired degree of efflux administration when administered to a subject; and
(c) combining the at least one lipophilic compound and the one or more compounds of Formula I to form a composition.

20. At least one compound chosen from compounds of Formula II: 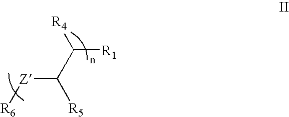
a pharmaceutically acceptable salt or ester thereof, a solvate thereof, a chelate thereof, a non-covalent complex thereof, a prodrug thereof, and mixtures of any of the foregoing, wherein:
n is a number from 1 to 900, wherein the individual units may be the same or different;
each of R₄ and R₅ is independently chosen from —H, alkyl, substituted alkyl, cycloalkyl, substituted cycloalkyl, aryl, substituted aryl, aralkyl and substituted aralkyl;
Z′ is chosen from —O—, —N—, —NO—, —NR₄—, —S—, —SO— and —SO₂—, wherein R₄ is defined as above;
R₁ is chosen from a tocopherol, a steroid and a flavonoid; and R₆ is chosen from any R₁, alkyl, substituted alkyl, cycloalkyl, substituted cycloalkyl, aryl, substituted aryl, aralkyl and substituted aralkyl.
